SEQUENCE LISTING (27 sequences)

SEQ ID NO: 1 - protein, 15 aa, Artificial Sequence
  Synthetic oligopeptide
MHGDTPTLHEYMLDL

SEQ ID NO: 2 - protein, 15 aa, Artificial Sequence
  Synthetic oligopeptide
TPTLHEYMLDLQPET

SEQ ID NO: 3 - protein, 15 aa, Artificial Sequence
  Synthetic oligopeptide
HEYMLDLQPETTDLY

SEQ ID NO: 4 - protein, 15 aa, Artificial Sequence
  Synthetic oligopeptide
LDLQPETTDLYCYEQ

SEQ ID NO: 5 - protein, 15 aa, Artificial Sequence
  Synthetic oligopeptide
PETTDLYCYEQLNDS

SEQ ID NO: 6 - protein, 15 aa, Artificial Sequence
  Synthetic oligopeptide
DLYCYEQLNDSSEEE

SEQ ID NO: 7 - protein, 15 aa, Artificial Sequence
  Synthetic oligopeptide
YEQLNDSSEEEDEID

SEQ ID NO: 8 - protein, 15 aa, Artificial Sequence
  Synthetic oligopeptide
NDSSEEEDEIDGPAG

SEQ ID NO: 9 - protein, 15 aa, Artificial Sequence
  Synthetic oligopeptide
EEEDEIDGPAGQAEP

SEQ ID NO: 10 - protein, 15 aa, Artificial Sequence
  Synthetic oligopeptide
EIDGPAGQAEPDRAH

SEQ ID NO: 11 - protein, 15 aa, Artificial Sequence
  Synthetic oligopeptide
PAGQAEPDRAHYNIV

SEQ ID NO: 12 - protein, 15 aa, Artificial Sequence
  Synthetic oligopeptide
AEPDRAHYNIVTFCC

SEQ ID NO: 13 - protein, 15 aa, Artificial Sequence
  Synthetic oligopeptide
RAHYNIVTFCCKCDS

SEQ ID NO: 14 - protein, 15 aa, Artificial Sequence
  Synthetic oligopeptide
NIVTFCCKCDSTLRL

SEQ ID NO: 15 - protein, 15 aa, Artificial Sequence
  Synthetic oligopeptide
FCCKCDSTLRLCVQS

SEQ ID NO: 16 - protein, 15 aa, Artificial Sequence
  Synthetic oligopeptide
CDSTLRLCVQSTHVD

SEQ ID NO: 17 - protein, 15 aa, Artificial Sequence
  Synthetic oligopeptide
LRLCVQSTHVDIRTL

SEQ ID NO: 18 - protein, 15 aa, Artificial Sequence
  Synthetic oligopeptide
VQSTHVDIRTLEDLL

SEQ ID NO: 19 - protein, 15 aa, Artificial Sequence
  Synthetic oligopeptide
HVDIRTLEDLLMGTL

SEQ ID NO: 20 - protein, 15 aa, Artificial Sequence
  Synthetic oligopeptide
RTLEDLLMGTLGIVC

SEQ ID NO: 21 - protein, 15 aa, Artificial Sequence
  Synthetic oligopeptide
DLLMGTLGIVCPICS

SEQ ID NO: 22 - protein, 14 aa, Artificial Sequence
  Synthetic oligopeptide
GTLGIVCPICSQKP

SEQ ID NO: 23 - DNA, 20 bp, Artificial Sequence
  Synthetic oligonucleotide
tccatgacgttcctgatgct

SEQ ID NO: 24 - DNA, 20 bp, Artificial Sequence
  Synthetic oligonucleotide
tccatgacgttcctgacgtt

SEQ ID NO: 25 - DNA, 20 bp, Artificial Sequence
  Synthetic oligonucleotide
tccatgacgttcctgatgct

SEQ ID NO: 26 - protein, 9 aa, Artificial Sequence
  Synthetic oligopeptide
RAHYNIVTF

SEQ ID NO: 27 - protein, 8 aa, Artificial Sequence
  Synthetic oligopeptide
MGLKFRQL

(19) United States
(12) Patent Application Publication
(10) Pub. No.: US 2011/0142873 A1
(43) Pub. Date: Jun. 16, 2011

(54) BIOACTIVE PURIFIED HSPE7 COMPOSITIONS

(76) Inventors: Gerry Rowse, Waxhaw, NC (US); John Webb, Sidney (CA); Marvin Siegel, Blue Bell, PA (US); Peter Emtage, San Diego, CA (US)

(21) Appl. No.: 12/160,880

(22) PCT Filed: May 30, 2007
(86) PCT No.: PCT/CA07/00963

§ 371 Date: Feb. 25, 2011

Related U.S. Application Data
(60) Provisional application No. 60/803,606, filed on May 31, 2006.

(51) Int. Cl. A61K 39/12; A61P 35/00; A61P 37/04; A61P 31/12

(52) U.S. Cl. 424/192.1

(57) ABSTRACT

A method of treating or preventing a condition related to an HPV infection is provided. The method comprises administering to a subject a composition comprising a purified Hsp65-E7 fusion protein (HspE7) admixed with an immune stimulant selected from the group consisting of CpG, a TLR3 agonist such as PolyI:C, PolyICLC, mono-phosphoryl-lipid A (MPL), MPL-trehalose 6,6′-dimycolate (MPL-TDM), and anti-CD40. A composition comprising HspE7 and one or more than one of CpG, a TLR3 agonist such as PolyI:C, PolyICLC, MPL, MPL-TDM, and anti-CD40, and method of reducing a tumor or virus development in a mammal or subject in need thereof by using the composition are also provided.

This application is a Continuation-in-Part of PCT Application No. PCT/CA2007/000963, filed May 30, 2007, which claims priority to U.S. Provisional Application No. 60/803,606 filed May 31, 2006.

### FIELD OF INVENTION

The present invention relates to the field of immunology. Furthermore, the present invention provides compositions comprising HspE7 and methods of their use.

### BACKGROUND OF THE INVENTION

Vaccination and immunotherapy strategies are directed to manipulation of a series of intricately choreographed series of cellular interactions. Cellular interactions include immune surveillance, whereby antigen presenting cells (APCs) in general, and dendritic cells (DCs) in particular, encounter and take up antigen, generate peptide epitopes from the antigen, and load the epitopes into recognition clefts of molecules that are encoded by the major histocompatibility complex (MHC). After export to the DC surface, epitope-laden MHC molecules present the epitope-MHC complex to T cells and activate the T cells. Activated CD4+ T helper (Th) cells deliver chemokine and cytokine signals to other DCs, enabling them, in turn, to activate naïve CD8+ T cells, transforming these cells into antigen-specific cytotoxic T lymphocytes (CTL). Activated T-helper cells interact with B cells as well, providing them with molecular signals that control differentiation, clonal expansion, and definition of the antibody isotype that they will secrete in mounting the humoral response of adaptive immunity.

Vaccination and immunotherapy are attractive approaches for prophylaxis or therapy of a range of disorders such as certain infectious diseases or cancers. However, the success of such treatments is often limited by several shortcomings inherent to immunotherapeutic protocols for example, poor immunogenicity of the chosen cytotoxic T lymphocytes (CTL) epitope. The standard method to increase the immune response is to use an adjuvant that is separate from the immunogen, and typically mixed with the immunogen prior to use. Alum and incomplete Freund's adjuvant (IFA) are well known examples of adjuvants. Certain microbial natural products have also been shown to be useful as adjuvants. Common examples include lipopolysaccharide (LPS) from Gram negative bacteria, and bacterial cell wall glycopeptides, also known as murein or peptidoglycan (PG), from both Gram negative and Gram positive bacteria.

Microbial adjuvants are thought to exert their pro-inflammatory effects by activating pattern recognition receptors (PRRs) in mammalian cells. Mammalian surface receptors known as Toll-like receptors (TLRs) are one of the key receptor classes in the PRR system. Activation of a TLR triggers an intracellular signaling cascade that leads to induction of the transcription factors NFκB and AP1 which in turn stimulates expression of genes encoding pro-inflammatory mediators such as chemokines and certain cytokines. Eleven different TLR have been identified to date in humans and each TLR has the ability to recognize a unique subset of microbial compounds

For example, LPS is a ligand of TLR4 and peptidoglycan is a ligand of TLR2. TLRs can also form heterodimers having unique ligand specificities. For example, the macrophage-activating lipopeptide 2 (MALP-2) from mycoplasma is a ligand for TLR2/TLR6 heterodimers whereas the bacterial lipopeptide Pam3Cys-Ser-Lys(4) is a ligand for TLR1/TLR2 heterodimers.

The E7 protein of Human Papillomavirus (HPV) is a small (approximately 10,000 Mw), Zn-binding phosphoprotein that has oncogenic properties, likely due to its ability to bind to the retinoblastoma gene product Rb (a tumor suppressor binding to and inactivating transcription factor E2F). The transcription factor E2F controls transcription of a number of growth-related genes including those encoding thymidine kinase, c-myc, dihydrofolate reductase and DNA polymerase alpha. Rb-E2F complex formation prevents the expression of the latter genes in G0 and G1 phases, restricting their expression to the S phase where the Rb-E2F complexes are programmed to dissociate, liberating active transcription factor E2F. Thus E7 represents an attractive target for immunological intervention in papilloma virus infections as it is expressed throughout the virus lifecycle and indeed it is one of only two viral proteins expressed during late stage cervical carcinoma caused by HPV infection.

The co-administration of adjuvants with HPV 16 proteins has been reported. For example, Freyschmidt et al. (Freyschmidt E-J., et al., 2004, Antiviral Ther. 9:479-489) demonstrate that lipopolysaccharide (LPS), unmethylated CpG and sorbitol enhanced a HPV16 L1-E7 fusion particle-induced stimulation of dendritic cells. Kim et al. (Kim T-Y., et. al., 2002 Cancer Res. 62:7234-7240) teach that co-delivery of HPV E7 with CpG oligodeoxynucleotide (CpG ODN) 1826 increases protective immunity against HPV 16. Elimination of E5-containing tumor growth has also been reported by Chen et al. (Chen Y-F., et. el., 2004, J. Virol. 78:1333-1343) using HPV E5 co-administered with CpG ODN 1826 or Freunds adjuvant.

WO99/07860 discloses the preparation of a recombinant Hsp65-E7 fusion protein (HspE7) that is useful as a vaccine reagent for eliciting anti-E7 immune responses during HPV infection. The HspE7 fusion protein described therein is expressed in E. coli and is biologically active in terms of its ability to elicit E7-specific CD8 immune responses.

### SUMMARY OF THE INVENTION

The present invention relates to compositions comprising HspE7 and methods of their use. More specifically, the present invention provides compositions comprising purified Hsp65-HPV E7 fusion (HspE7), and methods for use.

It is an object of the invention to provide an improved HspE7 composition.

According to the present invention there is provided a method of increasing the biological activity of purified HspE7 comprising, admixing the HspE7 along with an immune stimulant selected from the group consisting of CpG, a TLR3 agonist, mono-phosphoryl-lipid A (MPL), MPL-trehalose 6,6′-dimycolate (MPL-TDM), and anti-CD40. Preferably the immune stimulant is co-administered with HspE7 at an amount from about 1 ug to about 5000 ug per dose. In some aspects of the invention, the immune stimulant is PolyI:C or polyI:C complexed with a cationic polymer such as poly-lysine, poly-arginine or a cationic peptide comprising a majority of cationic amino acids. In other aspects of the invention, the immune stimulant is PolyICLC. Furthermore, the purified HspE7 is of a purity of about 95% to about 99.99% as determined using gel electrophoresis, HPLC, or both.

The present invention is also directed to a composition comprising purified HspE7 and an immune stimulant selected from the group consisting of CpG, a TLR3 agonist, MPL, MPL-TDM and anti-CD40. Preferably the immune stimulant is present at an amount from about 1 ug to about 5000 ug per dose. In some aspects of the invention, the immune stimulant is PolyI:C or polyI:C complexed with a cationic polymer such as poly-lysine, poly-arginine or a cationic peptide comprising a majority of cationic amino acids. In other aspects of the invention, the immune stimulant is PolyICLC. Furthermore, the purified HspE7 is of a purity of about 95% to about 99.99% as determined using gel electrophoresis, HPLC, or both.

The present invention is also directed to a method of reducing tumor burden or viral development in a mammal or subject comprising, administering the composition comprising purified HspE7 and an immune stimulant selected from the group consisting of CpG, a TLR3 agonist, MPL, MPL-TDM and anti-CD40, to a subject in need thereof. Preferably the immune stimulant is co-administered at an amount from about 1 ug to about 5000 ug per dose. In some aspects of the invention, the immune stimulant is PolyI:C or polyI:C complexed with a cationic polymer such as poly-lysine, poly-arginine or a cationic peptide comprising a majority of cationic amino acids. In other aspects of the invention, the immune stimulant is PolyICLC. Furthermore, the purified HspE7 is of a purity of about 95% to about 99.99% as determined using gel electrophoresis, HPLC, or both.

The present invention further provides a kit comprising purified HspE7 and an immune stimulant selected from the group consisting of CpG, a TLR3 agonist, MPL, MPL-TDM and anti-CD40, and instruction for their use. Preferably the immune stimulant is present at an amount from about 1 ug to about 5000 ug/dose. In some aspects of the invention, the immune stimulant is PolyI:C or polyI:C complexed with a cationic polymer such as poly-lysine, poly-arginine or a cationic peptide comprising a majority of cationic amino acids. In other aspects of the invention, the immune stimulant is PolyICLC. Furthermore, the purified HspE7 is of a purity of about 95% to about 99.99% as determined using gel electrophoresis, HPLC, or both.

The present invention is also directed to a method of treating or preventing a condition related to an HPV infection in a subject, comprising administering a composition comprising purified HspE7 and an immune stimulant selected from the group consisting of CpG containing oligonucleotides, a TLR3 agonist, mono-phosphoryl-lipid A (MPL), MPL-trehalose 6,6′-dimycolate (MPL-TDM), and anti-CD40. In some aspects of the invention, the immune stimulant is PolyI:C or polyI:C complexed with a cationic polymer such as poly-lysine, poly-arginine or a cationic peptide comprising a majority of cationic amino acids. In other aspects of the invention, the immune stimulant is PolyICLC. Furthermore, the purified HspE7 is of a purity of about 95% to about 99.99% as determined using gel electrophoresis, HPLC, or both.

The present invention relates to uses of the compositions to enhance immune responses against HPV protein antigens and, in particular embodiments, against tumors or HPV-infected cells expressing an HPV protein antigen. The compositions may be used in the prevention or treatment of cancer in a subject in need thereof.

The present invention also relates to a dosing schedule for the compositions. In particular aspects of the invention, compositions of the present invention are administered in a dosing schedule comprising at least two doses. The doses may be administered on consecutive days, or on non-consecutive days, or a combination thereof.

In some aspects of the invention, the condition related to an HPV infection is cervical intraepithelial neoplasia, bowenoid papulosis, Buschke-Lowenstein tumor, Butcher's/meat handlers warts, cutaneous squamous cell carcinoma, Epidermodysplasia verruciformis, Keratoacanthoma, Oral focal epithelia hyperplasia, Heck's disease, warts in renal transplant patients, common warts (verrucae vulgaris), filiform warts, flat warts, plantar, palmar or mosaic warts, periungual warts, refractory warts, genital warts, condyloma, condylomata acuminata, venereal warts, cutaneous papillomavirus disease, squamous cell papilloma, transitional cell papilloma, or bladder papilloma. In some aspects of the invention, the HPV infection may include an HPV of one or more types 1-5, 6, 7, 8, 9 10, 11, 12, 13, 14, 15, 16, 17, 18, 19, 20 31, 32, 33, 35, 36, 38, 39, 41, 47, 48, 50 or 75-77.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows anti-tumor activity of various HspE7 preparations. Process L: Process L HspE7, is a highly purified HspE7 preparation). Process A HspE7 is a less pure HspE7 (described in WO 99/07860). Mice bearing established E7-expressing TC-1 tumors were injected subcutaneously in the scruff of the neck with graded doses of HspE7 produced by either process A or process L (n=30/grp/dose) and followed for tumor growth for 49 days. RD4—Process L HspE7 (▾); RD5—Process L HspE7 (♦); CL4—Process A HspE7 (∘); CL6—Process A HspE7 (□). X axis: dose of HspE7 used in TC-1 assay in ug.

FIG. 2 shows augmentation of the ability of HspE7 to induce E7-specific CD8-positive T lymphocytes in the presence of a CpG-containing oligonucleotide (aTLR9 agonist). Naïve C57Bl/6 mice were injected subcutaneously with either HspE7 alone, or HspE7 plus 30 ug of CpG oligonucleotide and the number of E7-specific splenocytes was measured by ELISPOT. From left to right (cohorts of two mice per treatment), the immunizing antigen was 400 ug Process A HspE7 (less pure HspE7 described in WO 99/07860); 400 ug Process A HspE7 plus 30 ug CpG oligonucleotide; 400 ug Process L HspE7 (highly purified HspE7); 400 ug Process L HspE7 plus 30 ug CpG oligonucleotide The recall antigens used for ELISPOT analysis were HBV core antigen (HBVcAg) (93-100) irrelevant control peptide (solid bar); E7 (49-57) specific peptide (grey bar); medium only control (open bar).

FIG. 3 shows augmentation of the ability of HspE7 to induce E7-specific CD8-positive T lymphocytes by co-injection of Process L HspE7 (purified HspE7) with Poly I:C (TLR3 agonist) or CpG oligonucleotide (TLR9 agonist) but not PAM3CysSK4 (TLR2 agonist). Mice were injected subcutaneously with a mixture (solution) of Process L HspE7 plus TLR agonist at the indicated doses and the number of E7-specific splenocytes was measured by ELISPOT. From left to right (cohorts of two mice per treatment), immunizing antigen was 50 ug Process L HspE7 plus 10 ug CpG oligonucleotide; 50 ug Process L HspE7 plus 100 ug polyI:C; 50 ug Process L HspE7 plus 20 ug Pam3CysSK4;or naïve mice. The recall antigens used for ELISPOT analysis were HBVcAg (93-100) irrelevant control peptide (hatched bar); E7 (49-57) specific peptide (solid bar); medium only control (open bar).

FIG. 4 shows augmentation of the ability of Process L HspE7 to induce E7-specific CD8-positive T lymphocytes in the presence of mono-phosphoryl-lipid A (MPL; a TLR4 agonist). Naïve C57Bl/6 mice were injected subcutaneously with either Process L HspE7 alone (purified HspE7), or HspE7 plus MPL+TDM and the number of E7-specific splenocytes was measured by ELISPOT. From left to right (cohorts of two mice per treatment), immunizing antigen was 400 ug Process L HspE7; 400 ug Process L HspE7 in MPL+TDM (Ribi) or naïve mice. The recall antigens used for ELISPOT analysis were HBVcAg (93-100) irrelevant control peptide (solid bar); E7 (49-57) specific peptide (hatched bar); medium only control (open bar).

FIG. 5 shows augmentation of the ability of Process L HspE7 to induce E7-specific CD8-positive T lymphocytes in the presence of Poly ICLC (a TLR3 agonist). Naïve C57Bl/6 mice were injected subcutaneously with either Process L HspE7 alone (purified HspE7), or HspE7 plus graded doses of Poly ICLC and the number of E7-specific splenocytes was measured by ELISPOT. From left to right (cohorts of two mice per treatment), immunizing antigen was 400 ug Process L HspE7; 400 ug Process L HspE7 plus 100 ug Poly ICLC; 400 ug Process L HspE7 plus bug Poly ICLC; 400 ug Process L HspE7 plus 1 ug Poly ICLC; 400 ug Process L HspE7 plus 0.1 ug Poly ICLC; 100 ug Poly ICLC only or naïve mice. The recall antigens used for ELISPOT analysis were HBVcAg (93-100) irrelevant control peptide (grey bar); E7 (49-57) specific peptide (stippled bar); medium only control (open bar).

FIG. 6 shows the effect of Process L HspE7 or Process A HspE7 on tumor incidence. Anti-tumor activity of various HspE7 preparations was determined by administering Process A HspE7 (a less pure HspE7, described in WO 99/07860), or co-administering Process L HspE7 (purified HspE7) and CpG oligonucleotides. Mice bearing established E7-expressing TC-1 tumors were injected subcutaneously in the scruff of the neck with Process A HspE7 alone or graded doses of Process L HspE7 mixed with different doses of CpG oligonucleotide (n=30/grp) and followed for tumor growth for 49 days. 3 ug CpG oligonucleotide plus Process L HspE7 (▪); 10 ug CpG oligonucleotide plus Process L HspE7 (▴); 30 ug CpG oligonucleotide+Process L HspE7 (▾); Process A HspE7 (♦); Average process A HspE7 historical (∘). X axis—ug of HspE7 used in TC-1 assay.

FIG. 7 shows an increase in the anti-tumor activity of Process L HspE7 by combining PolyI:C with Process L HspE7 (purified HspE7). Mice bearing established E7-expressing TC-1 tumors were subcutaneously injected in the scruff of the neck with graded doses of Process L HspE7 alone or Process L HspE7 combined with PolyI:C (n=20/grp) and followed for tumor growth for 49 days. Approximately 50 percent of mice injected with 800 ug of Process L HspE7 had tumors on day 49. HspE7 (▪); HspE7+PolyIC (▴). X axis—ug of HspE7 used in TC-1 assay.

FIG. 8 shows the effect of the adjuvants alum, or Freunds Incomplete Adjuvant (IFA), mixed with purified HspE7 (Process L HspE7), on inducing E7-specific, CD8-positive T lymphocytes. Mice were injected subcutaneously with either Process L HspE7 alone or with various combinations of Process L HspE7, CpG oligonucleotide, Alum and Freunds Incomplete Adjuvant (IFA) at the indicated doses and the number of E7-specific splenocytes was measured by ELISPOT. From left to right (cohorts of two mice per treatment), immunizing antigen was 400 ug Process L HspE7; 400 ug Process L HspE7 in IFA; 400 ug Process L HspE7 plus CpG oligonucleotide in IFA; 400 ug Process L HspE7 plus Alum; 400 ug Process L HspE7 plus Alum plus CpG oligonucleotide; 400 ug Process L HspE7 plus CpG oligonucleotide or naive. The recall antigens used for ELISPOT analysis were HBVcAg (93-100) irrelevant control peptide (hatched bar); E7 (49-57) specific peptide (stippled bar); medium only control (open bar).

FIG. 9 shows a comparison of the ability of HspE7 to induce E7-specific CD8-positive T lymphocytes when co-administered in the presence of various TLR agonists or an agonistic anti-CD40 antibody. Negligible numbers of E7-specific T cells were elicited after co-administration of HspE7 with Imiquimod (TLR7 agonist), PAM3CysSK4 (TLR1/2 agonist) or LPS (TLR4 agonist). In contrast large numbers of E7-specific T cells were elicited after co-administration of HspE7 with CpG oligonucleotide or agonistic anti-CD40 antibody. Mice were injected subcutaneously with a mixture of purified HspE7 (Process L HspE7) plus the indicated TLR-agonist, and the number of E7-specific splenocytes was measured by ELISPOT. From left to right (cohorts of two mice per treatment), immunizing antigen was 400 ug Process L HspE7; 400 ug Process L HspE7 plus 100 ug imiquimod; 400 ug Process L HspE7 plus 30 ug LPS; 400 ug Process L HspE7 plus 25 ug PAM3CysSK4; 400 ug Process L HspE7 plus 25 ug anti-CD40 antibody (clone 1C10); 400 ug Process L HspE7 plus 30 ug CpG oligonucleotide; or naïve mice. The recall antigens used for ELISPOT analysis were HBVcAg (93-100) irrelevant control peptide (solid bar); E7 (49-57) specific peptide (hatched bar); medium only control (open bar).

FIG. 10 shows the effect of a daily prime boost strategy on the ability to elicit class I-restricted CD8+ T cell responses as measured by—IFN-gamma ELISPOT. C57Bl/6 mice (2 per group) were immunized with HspE7 (100 ug) and polyICLC (10 ug) at daily intervals, once per day up to a maximum of 4 days. 7 days after the first exposure to antigen, all animals were euthanized and their splenocytes taken for analysis. IFN-gamma ELISPOT was used to assess the class 1-restricted CD8+ T-cell response upon stimulation with 16E7.49-57.Db peptide (Recall antigen—open bar; media-only control—solid bar). From left to right (cohorts of two mice per treatment) immunizing antigen was: 400 μg Process L HspE7 with 40 μg polyICLC (one dose); 100 μg Process L HspE7 with 10 μg polyICLC (one dose); 100 μg Process L HspE7 with 10 μg polyICLC (two doses); 100 μg Process L HspE7 with 10 μg polyICLC (three doses); 100 μg Process L HspE7 with 10 μg polyICLC (four doses); naïve mice.

FIG. 11 shows the effect of co-immunization of HspE7 plus Poly-ICLC on humoral immunity. Groups of C57Bl/6 mice (n=5) were immunized twice at monthly intervals (day 1, 28) with (from left to right on X axis) buffer, 500 μg HspE7, 12.5 μg Poly-ICLC, 500 μg HspE7+1.25 μg Poly-ICLC, 500 μg HspE7+12.5 μg Poly-ICLC or 500 μg HspE7+125 μg Poly-ICLC. Blood samples were taken for analysis of serum antibodies 7 days prior to dosing (d-7, baseline), and at day 21, 49 and 77). Sera from individual mice were tested for the presence of antibodies (IgG1, IgG2b and IgG2c) to E7 and HspE7 by standard ELISA. Data are expressed as the highest dilution of sera that gave an absorbance greater than the assay plate background (defined as 0.2 OD units). Panel A) Anti-E7 IgG1 titers; B) Anti-HspE7 IgG1 titers; C) Anti-E7 IgG2b titers; D) Anti-HspE7 IgG2b titers; E) Anti-E7 IgG2c titers; F) Anti-HspE7 IgG2c titers. Open bar—pre-bleed control; hatched bar—day 21 bleed; solid bar—day 49 bleed; striped bar—day 77 bleed.

FIG. 12 shows the results of immunization with exogenous antigen plus polyICLC in eliciting an antigen-specific CD8⁺ T cell responses. C57Bl/6 mice (two mice per cohort) were immunized subcutaneously with 400 μg HspE7 alone, 400 μg HspE7 with 100 μg polyICLC, 400 μg HspE7 with 10 ug polyICLC, 400 μg HspE7 with 1 ug polyICLC, 400 μg HspE7 with 0.1 ug polyICLC, 100 ug polyICLC alone, or buffer (control). Seven days post-immunization, antigen-specific CD8 T cell responses against the H-2D^{b} restricted epitope E7₄₉₋₅₇ were evaluated by IFN-gamma ELISPOT. Recall antigen for ELISPOT: open bar—media control; grey bar E7 peptide; black bar—HBVCor peptide.

FIG. 13 shows the results of a multiple-dose immunization with HspE7 plus polyICLC in inducing regression of large, established TC-1 tumors. C57Bl/6 mice (15 mice per cohort) were implanted with E7-expressing TC-1.K tumor cells (6×10⁴) on day 0 and were treated with 4 consecutive daily doses of buffer only (open square); 100 ug HspE7 protein (open triangle); 10 ug PolyICLC (open circle); or 100 ug HspE7 protein+10 ug PolyICLC (solid circle), starting on day 28 post-implantation. Data are presented as the median tumor volume for each cohort over time (panel A) or as tumor volume over time for individual animals within each cohort (panel B).

FIG. 14 shows the results of multiple-dose immunization strategies using HspE7 antigen plus TLR3 agonists. (A) C57Bl/6 mice (two mice per cohort) were immunized s.c. with recombinant HspE7 protein (100 ug) plus the TLR3 agonist PolyICLC (10 ug) either once at day 0 (solid square), twice at days 0 and 2 (open square) or twice at days 0 and 4 (solid circle). At the indicated time point (days after the first immunization) antigen-specific CD8 T cell responses against the H-2D^{b} restricted epitope E7₄₉₋₅₇ were evaluated by IFN-gamma ELISPOT. (B) C57Bl/6 mice (four mice per cohort) were immunized s.c. with recombinant HspE7 protein (100 ug) plus the TLR3 agonist PolyICLC (10 ug) together every day for four consecutive days, together once at day 1, or together once at day 1 followed by PolyICLC (10 ug) only on days 2, 3 and 4. Seven days after the first immunization, antigen-specific CD8 T cell responses against the H-2D^{b} restricted epitope E7₄₉₋₅₇ were evaluated by IFN-gamma ELISPOT.

FIG. 15 shows the results of multiple-dose immunization strategies using HspE7 antigen plus TLR3 agonists. (A) C57Bl/6 mice (two mice per cohort) were immunized daily for the indicated number of consecutive days with recombinant HspE7 protein (100 ug) plus PolyICLC (10 ug), or with a single dose of HspE7 protein (400 ug) plus PolyICLC (40 ug). Seven days after the first immunization, antigen-specific CD8 T cell responses against the H-2D^{b} restricted epitope E7₄₉₋₅₇ were evaluated by IFN-gamma ELISPOT. (B) Splenocytes from naïve mice (right panel) or mice receiving four consecutive daily doses of HspE7 protein (100 ug) plus PolyICLC (10 ug) (left panel) were stained with PE-conjugated H-2D^{b} pentamers (Proimmune) loaded with the E7₄₉₋₅₇ peptide and surface stained with anti-CD8 and anti-CD44 mAbs. Cells shown represent gated CD8⁺ positive populations. (C) C57Bl/6 mice (two mice per cohort) were immunized daily for the indicated number of consecutive days with recombinant HspE7 protein (100 ug) plus PolyICLC (10 ug). At the indicated time point (days after the first immunization) antigen-specific CD8 T cell responses against the H-2D^{b} restricted epitope E7₄₉₋₅₇ were evaluated by IFN-gamma ELISPOT.

FIG. 16 shows the Anti-HspE7 Antibody titer in patients from cohorts 1 and 2. Cohort 1 had 4 subjects receiving 500 ug of hspE7 in combination with 50 ug polyICLC; cohort 2 had 4 subjects receiving 500 ug of HspE7 in combination with 500 ug polyICLC. “Pre-immune” samples were collected from patients before starting the immunization protocol, and 7 days after each of the three administrations of the HspE7 and polyICLC dose. Pre-immune and study exit (final sample taken 7 days after the 3^{rd} dose) results are shown. X-axis is patient identification (cohort/subject); Y axis is anti-HspE7 antibody titer. C1—cohort 1; C2—cohort 2; P1—patient 1; P2—patient 2; P3—patient 3; P4—patient 4.

FIG. 17 shows the HPV 16 E7 specific CD8+ T cell responses for cohort 2. Cohort 2 had 4 subjects, each of whom received 3 doses of 500 ug HspE7+500 ug polyICLC. X-axis is patient and sample identification (cohort/subject/dose); Y axis is spots per 10⁶ PBMC. C2—cohort 2; P1—patient 1; P2—patient 2; P3—patient 3; P4—patient 4.

FIG. 18 shows the HPV 16 E7 specific CD8+ T cell responses for cohort 3. Cohort 3 had 4 subjects, each of whom received 3 doses of 500 ug HspE7+1000 ug polyICLC. X-axis is patient and sample identification (cohort/subject/dose); Y axis is spots per 10⁶ PBMC. C3—cohort 3; P1—patient 1; P2—patient 2; P3—patient 3; P4—patient 4.

FIG. 19 shows the HPV 16 E7 specific CD4+ T cell responses for cohort 3. Cohort 3 had 4 subjects, each of whom received 3 doses of 500 ug HspE7+1000 ug polyICLC.CD4+ T-cell response. X-axis is patient and sample identification (cohort/subject/dose); Y axis is ³H thymidine uptake (cpm). C3—cohort 3; P1—patient 1; P2—patient 2; P3—patient 3; P4—patient 4.

### DETAILED DESCRIPTION

The present invention relates to compositions comprising HspE7 and methods of their use.

The following description is of a preferred embodiment.

The present invention provides a composition comprising a purified HspE7 along with an immune stimulant, such as but not limited to a TLR agonist, and optionally, other pharmaceutically acceptable ingredients. The immune stimulant may be a TLR3, or a TLR9 agonist, however, other TLR agonists may also be employed. Examples of immune stimulants that may be admixed with the purified HspE7 include, but are not limited to, CpG-containing oligonucleotides (a TLR9 agonist), A TLR3 agonist for example double-stranded RNA(dsRNA) or PolyI:C, or PolyI:C with poly-L-lysine (polyICLC), mono-phosphoryl-lipid A (MPL; a TLR4 agonist) or MPL-trehalose 6,6′-dimycolate (MPL-TDM), and anti-CD-40.

By purified HspE7 it is meant an HspE7 preparation that is characterized as comprising from about 95% to about 99.99% HspE7 or any amount there between, with the remaining constituents comprising components that are present following HspE7 preparation and purification. For example the purified HspE7 may be characterized as comprising from about 95% to about 98%, or any amount there between, or from about 97 to about 99.6%, or any amount there between HspE7. A purified HspE7 may comprise about 95, 96, 97, 98, 99, 99.2, 99.4, 99.6, 99.8, 99.9, 99.95, 99.99% HspE7, or any amount there between. An example of a purified HspE7 is Process L HspE7.

The purity of HspE7, or Process L HspE7 may be determined using any known methods for purity evaluation including for example, but not limited to HPLC, or gel electrophoresis. For example, a combination of reducing and non-reducing gel electrophoresis (1% PAGE with SDS, ±beta-mercaptoethanol) as would be known to one of skill in the art.

The Hsp65-HPV E7 fusion product (HspE7) may be produced according to a variety of methods, for example, as disclosed in WO99/07860 (which is incorporated herein by reference). For use as described herein, the HspE7 preparation is followed by further purification. Further purification may be achieved using any known purification methods including chromatography, using one or more of size exclusion, ion-exchange (cation, anion or both), affinity, reverse phase, or other methods of chromatography, gel electrophoresis, either by size, charge or both, denaturation using chaotrope reagents for example but not limited to urea or guanidine hydrochloride, salt or pH precipitation, membrane filtration, and the like as would be known to one of skill in the art.

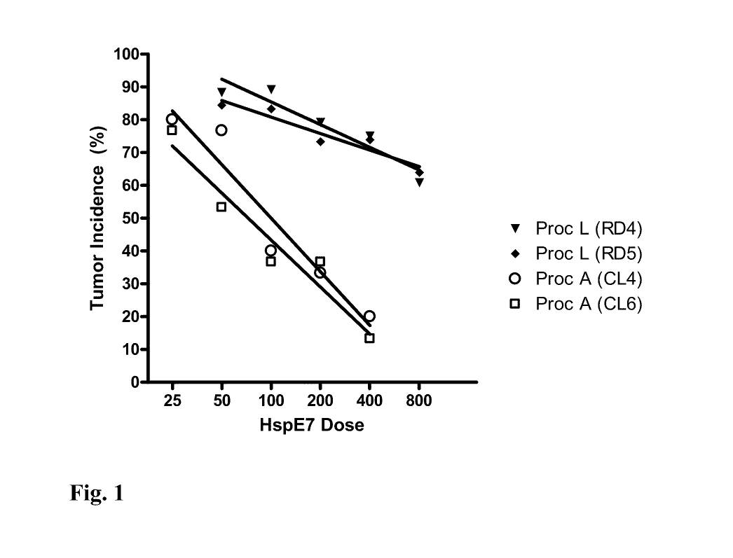

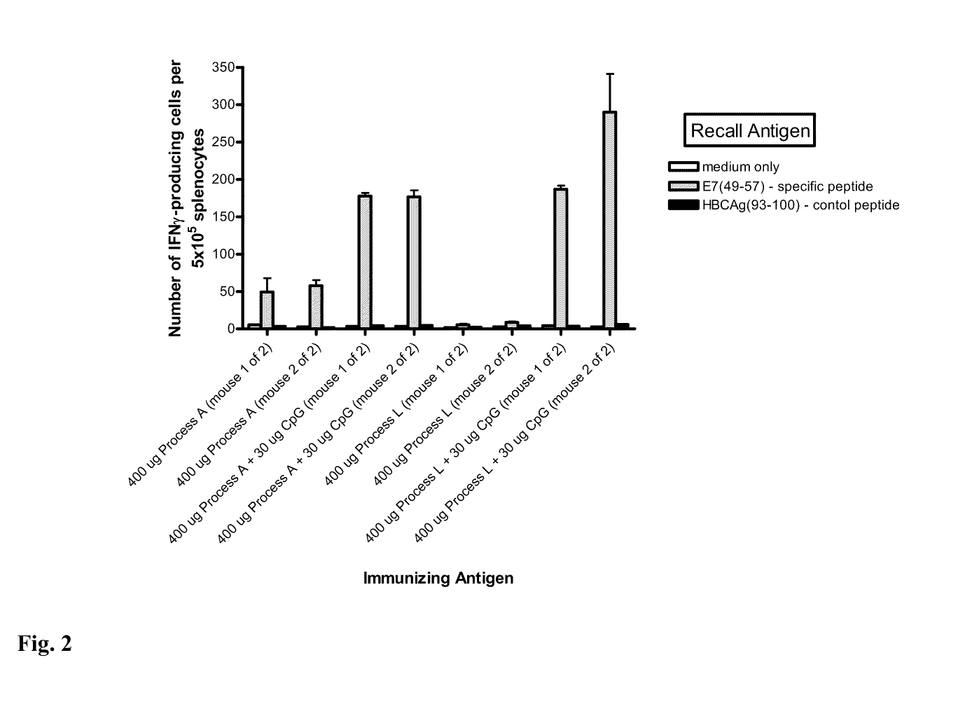

The HspE7 disclosed in WO99/07860 is a less-pure preparation, for example, comprising a purity less than about 95%, than the highly purified HspE7 (Process L HspE7) described herein. The less pure form of HspE7 is referred to as Process A HspE7, or Process A. Without wishing to be bound by theory, Process A HspE7 comprises one or more than one component that results in its enhanced biological activity when compared to a more purified HspE7, for example Process L HspE7 (e.g. see FIGS. 1 and 2). However, as described herein, when the prior art HspE7 (Process A HspE7) is further purified to produce a low toxicity HspE7, to a purity of between about 95% to about 99.99%, or any amount there between (Process L HspE7), a loss in biological activity of the HspE7 is observed (see FIGS. 1 and 2; Process L HspE7 v. Process A HspE7; and Examples 2 and 3). As shown in FIG. 1, the use of Process L HspE7 (purified HspE7) does not exhibit as significant a reduction in tumor incidence as observed using less pure, Process A HspE7, over a similar dose range. However, as described below, the highly purified HspE7, for example but not limited to Process L HspE7, exhibits biological activity when co-administered with an immune stimulant, such as but not limited to a TLR agonist. The purified HspE7 composition comprising purified HspE7 and an immune stimulant may further comprise other pharmaceutically acceptable ingredients. The immune stimulant may be a TLR3, or a TLR9 agonist, however, other TLR agonists or adjuvants, for example CD40 may also be employed.

Examples of immune stimulants that may be admixed with the purified HspE7 include, but are not limited to, CpG-containing oligonucleotides (a TLR9 agonist), PolyI:C, PolyICLC (TLR3 agonists), mono-phosphoryl-lipid A (MPL; a TLR4 agonist), MPL-trehalose 6,6′-dimycolate (MPL-TDM), and anti-CD-40 antibody. Non-limiting examples of CpG oligonucleotides may include for example CpG's comprising a class B type core sequence: GACGTT, for example which is not to be considered limiting CpG 1982, 1826, or 1668. CpG 1982 has the following sequence: TCC ATG ACG TTC CTG ATG CT (SEQ ID NO:1). CpG 1982 is available with a phosphorothioate backbone (from Invitrogen, and is designated: ZOO FZE FOE ZZO OZE FZE OT). CpG 1826 has the following sequence: TCC ATG ACG TTC CTG ACG TT (SEQ ID NO:2). CpG 1668 comprises the sequence: TCC ATG ACG TTC CTG ATG CT (SEQ ID NO:3). Preferably the CpG oligonucleotides 1982 and 1668 comprise a phosphorothioate backbone. Several CpG-containing oligonucleotides with the optimal murine class B type core sequence (GACGTT), including 1668, have been shown to be highly active in augmenting the activity of Process L HspE7. Similarly a CpG class C type oligonucleotide (2395) was found to be highly active. However a class A type CpG-containing oligonucleotide was found to be far less effective in augmenting the activity of HspE7 in the ELISPOT assay. For an explanation of the A, B and C classes of CpG see Vollmer J., et al. (Vollmer J., et al., 2004, Eur J. Immunol. 34:251-262).

PolyIC ribonucleic acids, including double-stranded ribonucleic acids (dsRNA) combined with other agents have demonstrated improved stability profiles, for example reduced susceptibility to endogenous RNAses. The dsRNA may be, for example, encapsulated in lipid vesicles, or complexed with a polycationic polymer. Examples of such polymers include, but are not limited to peptides comprising a majority of cationic amino acids, poly-lysine, poly-arginine or the like, U.S. Pat. No. 4,346,538 describes polyIC complexes comprising relatively high molecular weight polyI:C, poly-L-lysine in a MW range of 13-35 kDa and carboxymethylcellulose (“polyICLC”); and methods of preparation and using such compositions. The use of polyICLC as a therapeutic agent for the treatment of some cancers, some viral diseases such as HIV or Ebola, and also in multiple sclerosis has also been suggested (US Publication 2006/0223742).

Double-stranded RNA polyIC ribonucleic acids may, in some embodiments, comprise a polyI oligonucleotide and a polyC oligonucleotide in an anti-parallel base-paired configuration. The strands of such double-stranded nucleic acid molecules, interact in an ordered manner through hydrogen bonding—also referred to as ‘Watson-Crick’ base pairing. Variant base-pairing may also occur through non-canonical hydrogen bonding includes Hoogsteen base pairing. Under some thermodynamic, ionic or pH conditions, triple helices may occur, particularly with ribonucleic acids. These and other variant hydrogen bonding or base-pairing are known in the art, and may be found in, for example, Lehninger—Principles of Biochemistry, 3^{rd} edition (Nelson and Cox, eds. Worth Publishers, New York.), herein incorporated by reference.

A “polyI” oligonucleotide includes a majority of inosine, inosine-analogue nucleosides, or a combination thereof. Inosine-analogue nucleosides include, for example, 7-Deazainosine, 2′-O-methyl-inosine, 7-thia-7,9-dideazainosine, formycin B, 8-Azainosine, 9-deazainosine, allopurinol riboside, 8-bromo-inosine, 8-chloroinosine and the like.

A “polyC” oligonucleotide includes a majority of cytidine, cytidine-analogue nucleosides, or a combination thereof Cytidine-analogue nucleosides include, for example, 5-methylcytidine, 2′-O-methyl-cytidine, 5-(1-propynyl)cytidine, and the like.

Nucleic acids comprising non-canonical nucleosides and/or internucleosidic linkages may also provide improved stability profiles when used as adjuvants, and give a modified immunostimulatory effect, or modify the biological activity of the HspE7 compositions described herein. “Canonical” nucleosides include naturally occurring nucleosides such as deoxyadenosine, deoxyguanosine, deoxythymidine, deoxyuridine, deoxycytidine, deoxyinosine, adenosine, guanosine, 5-methyluridine, uridine and cytidine. A modified immunostimulatory effect may manifest as a quicker response of the adaptive, innate or humoral immune response, or may be a longer lasting, but less immediate, response.

Examples of non-canonical nucleosides are widely known in the art, and include, for example, the ‘locked nucleic acids’ or ‘LNAs. An LNA is a nucleoside having a 2′-4′ cyclic linkage as described in WO 99/14226, WO 00/56746, WO 00/56748, WO 01/25248, WO 0148190, WO 02/28875, WO 03/006475, WO 03/09547, WO 2004/083430, U.S. Pat. No. 6,268,490, U.S. Pat. No. 6,79449, U.S. Pat. No. 7,034,133. Other non-LNA bicyclic nucleosides are also known in the art, for example:
bicyclo[3.3.0]nucleosides with an additional C-3′,C-5′-ethanobridge;
bicarbocyclo[3.1.0]nucleosides with an additional C-1′,C-6′- or C-6′,C-4′methano bridge
bicyclo[3.3.0]- and [4.3.0]nucleosides containing an additional C-2′,C-3′dioxalane ring synthesised as a dimer with an unmodified nucleoside where the additional ring is part of the internucleoside linkage replacing a natural phosphodiester linkage; dimers containing a bicyclo[3.1.0]nucleoside with a C-2′,C-3′-methano bridge as part of amide- and sulfonamide-type internucleoside linkages;
bicyclo[3.3.0]glucose derived nucleoside analogue incorporated in the middle of a trimer through formacetal internucleoside linkages;
tricyclo-DNA in which two five membered rings and one three membered ring constitute the backbone;
1,5-Anhydrohexitol nucleic acids; and
bicyclic[4.3.0]- and [3.3.0]nucleosides with additional C-2′,C-3′-connected six and five-membered ring.

Other non-canonical nucleosides and non-canonical internucleoside linkages (‘backbones’) that may be used in dsRNAs are described in, for example, Freier, 1997 (Nucleic Acids Res. 25:4429-4443) or Praseuth et al (Biochimica et Biophysica Acta 1489:181-206).

The purified HspE7 of the present invention is referred to as Process L HspE7 (or Process L). Without wishing to be bound by theory, one or more than one component may be removed from the HspE7 preparation during purification of the Process L HspE7, and the one or more than one component may impart an adjuvant-like activity to the less pure (Process A) HspE7 preparation. However, for clinical trials and regulatory approval of the HspE7 composition, the percent of unknown components within the composition needs to be minimized.

By biological activity of HspE7, it is meant any of the mediation, augmentation, or stimulation of an in vitro or in vivo biological activity by HspE7. Biological activity may also include inhibition of an in vitro or in vivo biological activity by HspE7. Many such activities are known and may be used as a basis for determining the biological activity of HspE7. For example, which is not to be considered limiting, the induction of E7-specific CD8-positive T lymphocytes may be used to determine the biological activity of HspE7. In one type of assay designed to measure this property (ELISPOT), the number of IFN-gamma producing cells per a given number of splenocytes is determined following treatment of a C57Bl/6 mouse with the compound or mixture of interest (see Example 2). An alternate assay involves determining the anti-tumor activity of HspE7 by treating mice with TC-1 tumors with a compound or mixture of interest, and determining the percent of tumor incidence after a period of time, for example a 49 day interval (see Example 2). Alternatively, stimulation of cytolytic activity (CTL assay) may also be used as would be known to one of skill in the art. Another non-limiting example of biological activity includes CD4-positive T lymphocyte stimulation. Such stimulation may be measured by a proliferation assay (see, for example, Example 13 and figures described therein). Biological activity may also include induction of a specific cell-mediated or humoral response to an immunogen or antigen, including production of specific antibodies of various types and subtypes.

The loss of activity resulting from the purification of HspE7 may be restored with the addition of an appropriate adjuvant or immune stimulant, such as but not limited to a TLR agonist to the HspE7 composition. To design-back the HspE7 composition, adjuvants were tested for their efficacy in restoring HspE7 activity. These adjuvants included CpG oligonucleotides, PolyI:C, PolyICLC, MPL, MPL-TDM, imiquimod, rough LPS (lipopolysaccharide), smooth LPS, Pam3CysSK4, anti-CD40, alum, and Freund's Incomplete Adjuvant (IFA). An immune stimulant such as PolyI:C or polyI:C may further be complexed with a cationic polymer such as poly-lysine, poly-arginine or a cationic peptide comprising a majority of cationic amino acids.

“Adjuvant” or an “immune stimulant” is a substance, or a combination of compounds that, when combined with an immunogen, enhances or augments the immune response against the immunogen. The enhancement or augmentation of an immune response may be determined using standard assays, including those described herein. An adjuvant or an immune stimulant may be comprised of one, or more than one compound.

“Immune response” means either a pro-inflammatory or anti-inflammatory response of the immune system, including the adaptive, humoral, innate and cell-mediated systems. The terms “modulate” or “modulation” or the like mean either an increase or a decrease in a selected parameter.

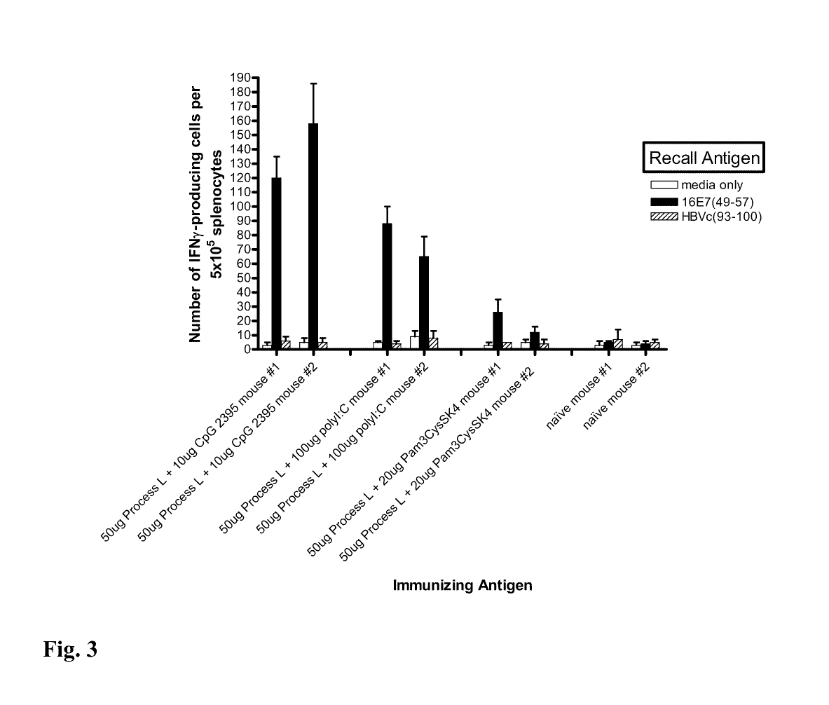

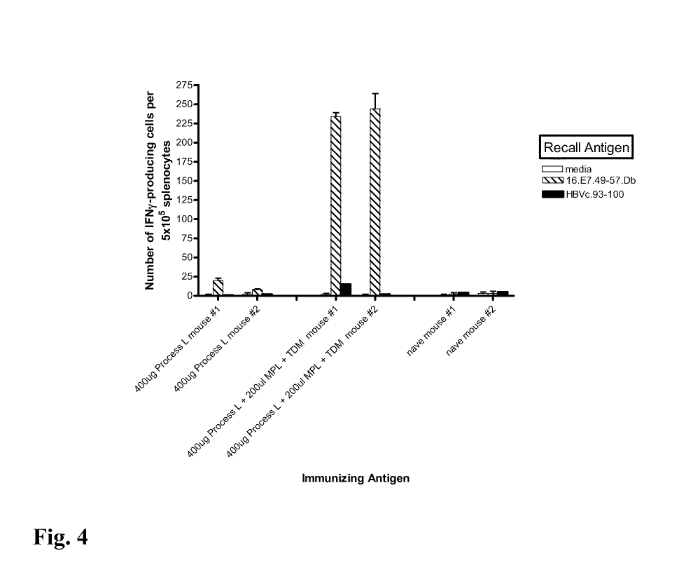

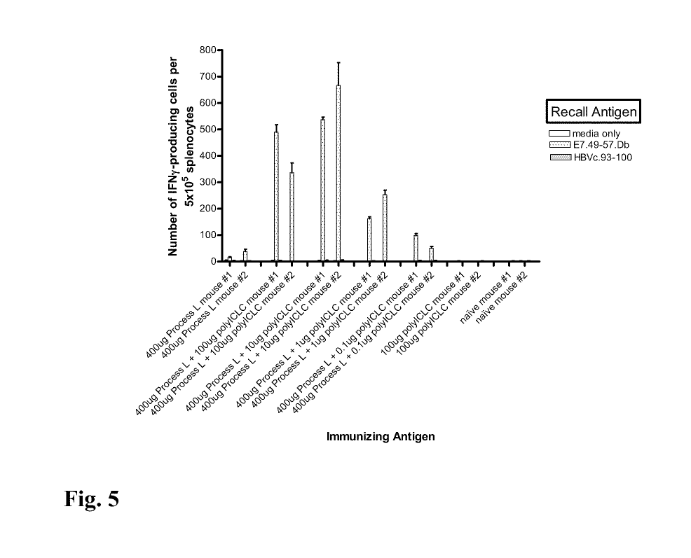

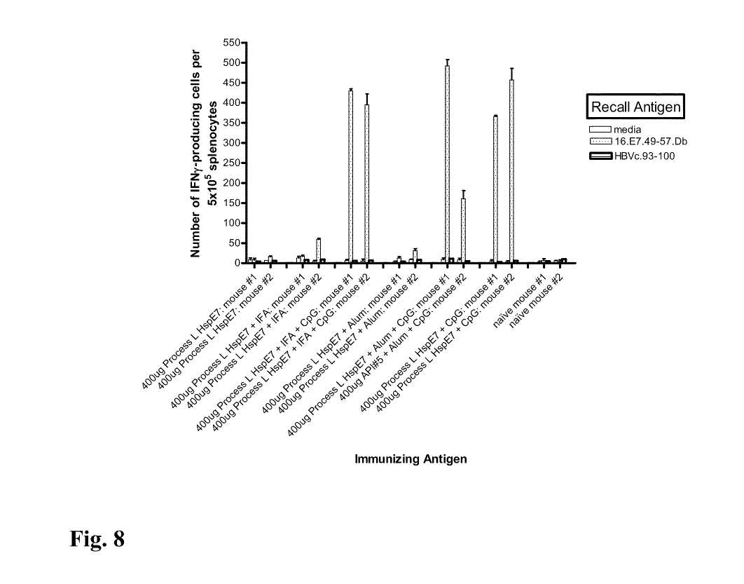

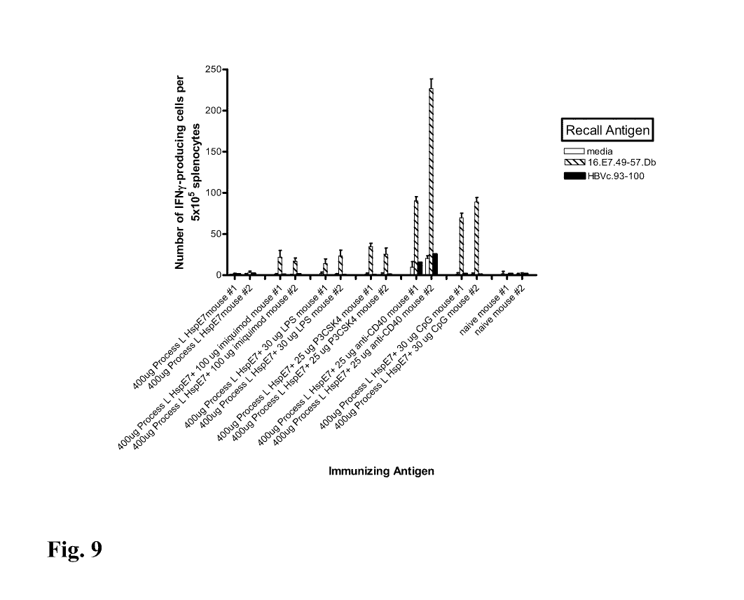

The addition of several well known adjuvants to purified HspE7, for example alum or Freunds Incomplete Adjuvant (IFA; see FIG. 8, Example 6), did not restore the loss of biological activity associated with HspE7 observed following purification, for example but not limited to, Process L HspE7. Similarly, the admixing of rough LPS (Example 7, FIG. 9), imiquimod (Example 7, FIG. 9), or Pam3CysSK4 (Example 7, FIG. 9), also did not augment HspE7 biological activity. However, the admixing of purified HspE7 with the CpG oligonucleotide (e.g. Example 3, FIGS. 2 and 3), PolyI:C (Example 4, FIG. 3), PolyICLC (FIG. 5), mono-phosphoryl-lipid A (MPL; FIG. 4), or anti-CD40 (FIG. 9) resulted in the restoration of biological activity associated with highly purified HspE7. The addition of CpG oligonucleotides, PolyI:C, or PolyICLC did not display this activity when administered in the absence of HspE7.

Therefore, the present invention also pertains to a method of increasing the biological activity of highly purified HspE7 comprising admixing or co-administering the purified HspE7 along with an immune stimulant selected from the group consisting of CpG oligonucleotides, PolyI:C, PolyICLC, mono-phosphoryl-lipid A (MPL), MPL-TDM, and anti-CD40. Preferably, the immune stimulant is present at an amount from about 0.1 ug to about 20 mg, or any amount therebetween, for example from about 1 ug to about 5000 ug/dose or any amount therebetween, about 10 ug to about 1000 ug or any amount therebetween, or about 30 ug to about 1000 ug or any amount therebetween. For example, a dose of about 0.1, 0.5, 1.0, 2.0, 5.0, 10.0 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, 5000, 10000, 20000 ug, or any amount therebetween may be used. Similarly, the purified HspE7 is present at an amount from about 0.1 ug to about 20 mg, or any amount therebetween, for example from about 1 ug to about 2000 ug/dose or any amount therebetween, about 10 ug to about 1000 ug or any amount therebetween, or about 30 ug to about 1000 ug or any amount therebetween. For example, a dose of about 0.1, 0.5, 1.0, 2.0, 5.0, 10.0 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, 5000, 10000, 20000 ug, or any amount therebetween may be used.

“Effective amount” refers to an amount of a compound or composition of the present invention effective to produce the desired or indicated immunologic or therapeutic effect. A non-limiting example of a dose to be achieved within a mammal or subject is about 0.3 mg/kg HspE7, immune stimulant, or both, and this can range from about 0.03 mg/kg to about 30.0 mg/kg HspE7, immune stimulant, or both, or any amount therebetween, as required. However, doses that are less than 0.03 mg/kg, or more than 30 mg/kg of HspE7, immune stimulant, or both may also be used and are also contemplated herein. One of skill in the art would be able to determine the appropriate dose of HspE7, immune stimulant, or both. For example a dose of about 0.1, 0.5, 1.0, 2.0, 5.0, 10.0 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, 5000, 10000, 20000 ug/kg, or any amount therebetween may be used. An immune stimulant such as PolyICLC or polyI:C may further be complexed or combined with a cationic polymer such as poly-lysine, poly-arginine or a cationic peptide comprising a majority of cationic amino acids. The cationic polymer may be combined with polyICLC or polyI:C in a ratio from about 10:1 to about 1:10.

Furthermore, the present invention provides a composition comprising purified HspE7 and an immune stimulant selected from the group consisting of CpG, a TLR3 agonist such as PolyI:C or PolyICLC, MPL, and anti-CD40. Preferably, the immune stimulant is present at an amount from about 0.1 ug to about 20 mg/dose, or any amount therebetween as defined above. For example, a dose of about 0.1, 0.5, 1.0, 2.0, 5.0, 10.0 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, 5000, 10000, 20000 ug, or any amount therebetween may be used.

The present invention also pertains to a method of reducing tumor growth in a subject, animal, or a patient comprising, administering a composition comprising purified HspE7 and an immune stimulant selected from the group consisting of CpG, a TLR3 agonist such as PolyI:C or PolyICLC, MPL, and anti-CD40. Preferably, the immune stimulant is present at an amount from about 0.1 ug to about 20 mg/dose, or any amount therebetween as defined above, to the subject, animal, or a patient in need thereof. For example a dose of about 0.1, 0.5, 1.0, 2.0, 5.0, 10.0 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, 5000, 10000, 20000 ug, or any amount therebetween may be used.

The terms “patient” or “subject” refer to mammals and other animals including humans and other primates, companion animals, zoo, and farm animals, including, but not limited to, cats, dogs, rodents, rats, mice, hamsters, rabbits, horses, cows, sheep, pigs, goats, poultry; etc.

Doses may be described as absolute quantity or concentration (e.g. 500 ug, or 500 ug/ml), or may be described with respect to the quantity relative to the mass of the subject or patient (e.g. 10 ug/kg). In some regimens, the mass of the subject is determined and a dose administered accordingly, alternately an ‘average’ subject or patient may be assumed for the purposes of calculation—e.g. an ‘average’ human subject of ˜70 kg, or an ‘average’ mouse of ˜35 grams. As an example, assuming a 70 kg human subject, a 50 ug dose may alternately be described as 0.7 ug/kg; a 500 ug does may be described as 7.1 ug/kg; a 1000 ug dose may be described as a 14.2 ug/kg dose; a 2000 ug dose may be described as a 28.5 ug/kg dose.

An immune stimulant such as PolyI:C or polyI:C may further be complexed with a cationic polymer such as poly-lysine, poly-arginine or a cationic peptide comprising a majority of cationic amino acids. Combining an immune stimulant with a cationic polymer may allow for a reduction in the effective amount necessary to produce the desired or indicated immunologic or therapeutic effect, relative to use of the immune stimulant in the absence of the cationic polymer. Alternately, combining an immune stimulant with a cationic polymer may allow for an altered dosing schedule necessary to produce the desired or indicated immunologic or therapeutic effect, relative to use of the immune stimulant in the absence of the cationic polymer, for example, a longer interval between doses in a dosing schedule, a fewer number of doses in a dosing schedule, or the like.

The HspE7 compositions of the present invention may be admixed with any suitable pharmaceutical carrier or salt. “Pharmaceutically acceptable salts” refers to the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-β hydroxynaphthoates, gentisates, isethionates, di-p-toluoyltartrates, methanesulphonates, ethanesulphonates, benzenesulphonates, p-toluenesulphonates, cyclohexylsulphamates and quinateslaurylsulphonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide. Suitable amine base addition salts are prepared from amines which have sufficient basicity to form a stable salt, and preferably include those amines which are frequently used in medicinal chemistry because of their low toxicity and acceptability for medical use, for example, ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

The HspE7 compositions of the present invention may be administered by any suitable route including injection, skin patch, or orally. Thus, in one aspect, the present invention provides pharmaceutical compositions for human and veterinary medical use comprising a compound comprising purified HspE7 admixed with an immune stimulant for example, anti-CD40, or a TLR agonist, including CpG, a TLR3 agonist such as PolyI:C or PolyICLC, or MPL, or a pharmaceutically acceptable salt thereof, together with one or more pharmaceutically or physiologically acceptable buffers, carriers, excipients, or diluents, and optionally, other therapeutic agents. It should be noted that compounds of the present invention can be administered individually, or in mixtures comprising two or more compounds. The present invention also encompasses the use of a compound comprising purified HspE7 admixed with a TLR agonist, including CpG, or PolyI:C, or a pharmaceutically acceptable salt thereof, for the preparation of a medicament for the prevention or treatment of an infection or pathology, or a disease state or condition in which an inflammatory immune response is beneficial.

The compounds of the present invention can be administered in pharmaceutically or physiologically acceptable solutions that can contain pharmaceutically or physiologically acceptable concentrations of salts, buffering agents, preservatives, compatible carriers, diluents, excipients, dispersing agents, etc., and optionally, other therapeutic ingredients. The compounds and compositions of the present invention can thus be formulated in a variety of standard pharmaceutically acceptable parenteral formulations as would be known to one of skill in the art.

The pharmaceutical compositions of the present invention can contain an effective amount of the presently disclosed compounds or compositions, optionally included in a pharmaceutically or physiologically acceptable buffer, carrier, excipient, or diluent. The term “pharmaceutically or physiologically acceptable buffer, carrier, excipient, or diluent” means one or more compatible solid or liquid fillers, dilutants, or encapsulating substances that are suitable for administration to a human or other animal. The term “carrier” denotes an organic or inorganic ingredient, natural or synthetic, with which the active ingredient is combined to facilitate the application. The components of the pharmaceutical compositions are capable of being commingled with the polymers of the present invention, and with each other, in a manner such that there is no interaction that would substantially impair the desired pharmaceutical efficiency of the active compound(s).

Compositions suitable for parenteral administration conveniently comprise sterile aqueous preparations, which can be isotonic with the blood of the recipient. Among the acceptable vehicles and solvents are water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. In addition, fatty acids such as oleic acid are useful in the preparation of injectables. Carrier formulations suitable for subcutaneous, intramuscular, intraperitoneal, intravenous, etc. administrations can be found in Remington: The Science and Practice of Pharmacy, 19th Edition, A. R. Gennaro, ed., Mack Publishing Co., Easton, Pa., (1995, which is incorporated herein by reference).

The compositions can be conveniently presented in unit dosage form or dosage unit form, and can be prepared by any of the methods well known in the art of pharmacy. All methods include the step of bringing the compound into association with a carrier that constitutes one or more accessory ingredients. In general, the compositions are prepared by uniformly and intimately bringing the compound into association with a liquid carrier, a finely divided solid carrier, or both. Compounds of the present invention can be stored lyophilized, and provided as a kit for admixing prior to use.

Other delivery systems can include time-release, delayed-release, or sustained-release delivery systems. Such systems can avoid repeated administrations of the compositions of the present invention, increasing convenience to the subject and the physician.

Microcapsules of the foregoing polymers containing drugs are described in, for example, U.S. Pat. No. 5,075,109. Delivery systems also include non-polymer systems such as: lipids, including sterols such as cholesterol, cholesterol esters, and fatty acids or neutral fats such as mono-, di-, and tri-glycerides; hydrogel release systems; silastic systems; peptide-based systems; wax coatings; compressed tablets using conventional binders and excipients; partially fused implants; and the like.

Determination of the optimal amount of compound to be administered to human or animal patients in need of prevention or treatment of chronic HPV infection or pathology associated with an HPV infection, or a disease or disorder which benefits from immune system stimulation, as well as methods of administering therapeutic or pharmaceutical compositions comprising such compounds, is well within the skill of those in the pharmaceutical, medical, and veterinary arts. Dosing of a human or animal patient is dependent on the nature of chronic HPV infection or pathology associated with an HPV infection or other disease or disorder to be treated, the patient's condition, body weight, general health, sex, diet, time, duration, and route of administration, rates of absorption, distribution, metabolism, and excretion of the compound, combination with other drugs, severity of the chronic HPV infection or pathology associated with an HPV infection or other disease or disorder to be treated, and the responsiveness of the pathology or disease state being treated, and can readily be optimized to obtain the desired level of effectiveness. The course of treatment can last from several days to several weeks or several months, or until a cure is effected or an acceptable diminution or prevention of the disease state is achieved. Optimal dosing schedules can be calculated from measurements of immune response in the body of the patient in conjunction with the effectiveness of the treatment. Persons of ordinary skill can easily determine optimum dosages, dosing methodologies, and repetition rates. Optimum dosages can vary depending on the potency of the immunomodulatory polymeric compound, and can generally be estimated based on ED₅₀ values found to be effective in in vitro and in vivo animal models. Effective amounts of the present compounds for the treatment or prevention of chronic HPV infection or pathology associated with an HPV infection or other diseases or disorders to be treated, delivery vehicles containing these compounds, agonists, and treatment protocols, can be determined by conventional means. For example, the medical or veterinary practitioner can commence treatment with a low dose of the compound in a first subject or patient, or first set of subjects or patients, and then increase the dosage, or systematically vary the dosage regimen in a second or subsequent subject or patient, or second or subsequent set of subjects or patients, monitor the effects thereof on the patients or subjects, and adjust the dosage or treatment regimen to maximize the desired therapeutic effect. Further discussion of optimization of dosage and treatment regimens can be found in Benet et al., in Goodman & Gilman's (1996, The Pharmacological Basis of Therapeutics, Ninth Edition, Hardman et al., Eds., McGraw-Hill, New York, Chapter 1, pp. 3-27; which is incorporated herein by reference) or Bauer (L. A. Bauer, 1999, in Pharmacotherapy, A Pathophysiologic Approach, Fourth Edition, DiPiro et al., Eds., Appleton & Lange, Stamford, Conn., Chapter 3, pp. 21-43; which is incorporated herein by reference).

A variety of administration routes are available. The particular mode selected will depend upon which compound is selected, the particular condition being treated, and the dosage required for therapeutic efficacy. Generally speaking, the methods of the present invention can be practiced using any mode of administration that is medically acceptable, meaning any mode that produces effective levels of an immune response without causing clinically unacceptable adverse effects. Preferred modes of administration are parenteral routes, although oral administration can also be employed. The term “parenteral” includes subcutaneous, intradermal, intravenous, intramuscular, or intraperitoneal injection, or infusion techniques.

In the context of the present invention, the terms “treatment,” “therapeutic use,” or “treatment regimen” as used herein are meant to encompass prophylactic, palliative, and therapeutic modalities of administration of the compositions of the present invention, and include any and all uses of the presently claimed compounds that remedy a disease state, condition, symptom, sign, or disorder caused by a chronic HPV infection or pathology associated with an HPV infection or other disease or disorder to be treated, or which prevents, hinders, retards, or reverses the progression of symptoms, signs, conditions, or disorders associated therewith. Thus, any prevention, amelioration, alleviation, reversal, or complete elimination of an undesirable disease state, symptom, condition, sign, or disorder associated with a chronic HPV infection or pathology associated with an HPV infection, or other disease or disorder that benefits from stimulation of the body's immune response, is encompassed by the present invention.

For purposes of the present invention, the meaning of the terms “treating,” “treatment,” and the like as applied to cancer therapy is broad, and includes a wide variety of different concepts generally accepted in the art. Thus, as used herein, this term includes, but is not limited to, prolongation of time to progressive disease; tumor reduction; disease remission; relief of suffering; improvement in life quality; extension of life; amelioration or control of symptoms such as pain, difficulty breathing, loss of appetite and weight loss, fatigue, weakness, depression and anxiety, confusion, etc.; improvement in patient comfort, etc. A separate goal may even be to cure the disease entirely.

The HspE7 of the present invention may be used to treat non-neoplasm, HPV-infected cells, or HPV induced disease states, for example but not limited to genital warts, hyperproliferative states, virally infected cells, chronically virally infected cells and the like.

The term “cancer” has many definitions. According to the American Cancer Society, cancer is a group of diseases characterized by uncontrolled growth (and sometimes spread) of abnormal cells. Although often referred to as a single condition, it actually consists of more than 200 different diseases. Cancerous growths can kill when such cells prevent normal function of vital organs, or spread throughout the body, damaging essential systems. The composition of the present invention may be used to treat susceptible neoplasms in an animal or subject in a method that comprises administering to the animal or subject in need thereof an effective amount of a compound or composition of the present invention.

Non-limiting examples of different types of cancers against which compounds of the present invention may be effective as therapeutic agents include: carcinomas, such as neoplasms of the central nervous system, including glioblastoma multiforme, astrocytoma, oligodendroglial tumors, ependymal and choroid plexus tumors, pineal tumors, neuronal tumors, medulloblastoma, schwannoma, meningioma, and meningeal sarcoma; neoplasms of the eye, including basal cell carcinoma, squamous cell carcinoma, melanoma, rhabdomyosarcoma, and retinoblastoma; neoplasms of the endocrine glands, including pituitary neoplasms, neoplasms of the thyroid, neoplasms of the adrenal cortex, neoplasms of the neuroendocrine system, neoplasms of the gastroenteropancreatic endocrine system, and neoplasms of the gonads; neoplasms of the head and neck, including head and neck cancer, neoplasms of the oral cavity, pharynx, and larynx, and odontogenic tumors; neoplasms of the thorax, including large cell lung carcinoma, small cell lung carcinoma, non-small cell lung carcinoma, malignant mesothelioma, thymomas, and primary germ cell tumors of the thorax; neoplasms of the alimentary canal, including neoplasms of the esophagus, stomach, liver, gallbladder, the exocrine pancreas, the small intestine, veriform appendix, and peritoneum, adneocarcinoma of the colon and rectum, and neoplasms of the anus; neoplasms of the genitourinary tract, including renal cell carcinoma, neoplasms of the renal pelvis, ureter, bladder, urethra, prostate, penis, testis; and female reproductive organs, including neoplasms of the vulva and vagina, cervix, adenocarcinoma of the uterine corpus, ovarian cancer, gynecologic sarcomas, and neoplasms of the breast; neoplasms of the skin, including basal cell carcinoma, squamous cell carcinoma, dermatofibrosarcoma, Merkel cell tumor, and malignant melanoma; neoplasms of the bone and soft tissue, including osteogenic sarcoma, malignant fibrous histiocytoma, chondrosarcoma, Ewing's sarcoma, primitive neuroectodermal tumor, and angiosarcoma; neoplasms of the hematopoietic system, including myelodysplastic syndromes, acute myeloid leukemia, chronic myeloid leukemia, acute lymphocytic leukemia, HTLV-1 and 5 T-cell leukemia/lymphoma, chronic lymphocytic leukemia, hairy cell leukemia, Hodgkin's disease, non-Hodgkin's lymphomas, and mast cell leukemia; and neoplasms of children, including acute lymphoblastic leukemia, acute myelocytic leukemias, neuroblastoma, bone tumors, rhabdomyosarcoma, lymphomas, and renal tumors.

PCT Patent Application WO 99/07860 provides, in addition to methods of making HspE7, a non-limiting discussion of various types of HPV and some of the pathologies that are caused by, linked with or associated with chronic HPV infection or pathology associated with an HPV infection. Other (non-limiting) examples of different types of chronic HPV infection or pathology associated with an HPV infection against which compounds of the present invention may be effective as therapeutic agents include: cervical intraepithelial neoplasia (for example, HPV types 16, 18, 31, 33, 35, 39), bowenoid papulosis (for example, HPV types 16, 18, 33, 39), Buschke-Lowenstein tumor (for example, HPV types 6, 11), Butcher's/meat handlers warts (for example, HPV type 7), cutaneous squamous cell carcinoma (for example, HPV types 38, 41, 48), Epidermodysplasia verruciformis (for example, HPV types 1-5, 7-9, 10, 12, 14, 15, 17-20, 23-25, 36, 47, 50), Keratoacanthoma (for example HPV type 77), Oral focal epithelia hyperplasia (Heck's disease) (for example, HPV types 13, 32), warts in renal transplant patients (for example, HPV types 75-77), common warts (verrucae vulgaris), filiform warts, flat warts, plantar, palmar or mosaic warts, periungual warts, refractory warts, genital warts, condyloma, condylomata acuminata, veneral warts, cutaneous papillomavirus disease, squamous cell papilloma, transitional cell papilloma (bladder papilloma), and the like.

A particular treatment regimen can last for a period of time which may vary depending upon the nature of the particular chronic HPV infection or pathology associated with an HPV infection or other disease or disorder to be treated, its severity, and the overall condition of the patient, and may involve administration of compound-containing compositions from once to several times daily for several days, weeks, months, or longer. Following treatment, the patient is monitored for changes in his/her condition and for alleviation of the symptoms, signs, or conditions of the disorder or disease state. The dosage of the composition can either be increased in the event the patient does not respond significantly to current dosage levels, or the dose can be decreased if an alleviation of the symptoms of the disorder or disease state is observed, or if the disorder or disease state has been ablated.

An optimal dosing schedule is used to deliver a therapeutically effective amount of the compounds of the present invention. For the purposes of the present invention, the terms “effective amount” or “therapeutically effective amount” with respect to the compounds disclosed herein refers to an amount of compound that is effective to achieve an intended purpose, preferably without undesirable side effects such as toxicity, irritation, or allergic response. Although individual patient needs may vary, determination of optimal ranges for effective amounts of pharmaceutical compositions is within the skill of the art. Human doses can be extrapolated from animal studies (A. S. Katocs, Remington: The Science and Practice of Pharmacy, 19th Ed., A. R. Gennaro, ed., Mack Publishing Co., Easton, Pa., (1995), Chapter 30; which is incorporated herein by reference). Generally, the dosage required to provide a therapeutically effective amount of a pharmaceutical composition, which can be adjusted by one skilled in the art, will vary depending on the age, health, physical condition, weight, type and extent of the disease or disorder of the recipient, frequency of treatment, the nature of concurrent therapy, and the nature and scope of the desired effect. Alternately, human doses may be determined empirically during a clinical study or trial

In some embodiments of the invention, a dosing schedule, alternately referred to as a treatment regimen, may comprise administration of an effective amount of a composition described herein over at least two, at least three, at least four, or more, days. For example, for a dosage schedule of four days (where day 0 (zero) is the day of the initial dose) the doses may be administered on consecutive days, or on non-consecutive days, or a combination thereof. In some examples, a dosing schedule may include administration on days 0 and 1; on days 0 and 2; on days 0 and 3; on days 0 and 4; on days 0, 1 and 2; on days 0, day 1 and 3; on days 0, 1 and 4; on days 0, 2 and 3; on days 0, 2 and 3; on days 0, 2 and 4; on days 0, 3 and 4; and the like.

A dosing schedule may have a longer period between doses, for example 1 week (about 7 days), 2 weeks (about 14 days), 3 weeks (about 21 days), 4 weeks (about 28 days), 5 weeks (about 35 days) or more, or any amount therebetween.

The amount of the dose may be the same, or about the same, for each dose of the dosing schedule, or it may be increased or decreased from the first dose for a subsequent dose.

In another embodiment, the dosing schedule may be effectively continuous, for example in a slow-release formulation that is administered via a dermal patch or by an implant.

Therefore, the invention provides for a method of treating or preventing a condition related to an HPV infection in a subject, comprising administering a composition comprising purified HspE7 and an immune stimulant selected from the group consisting of CpG containing oligonucleotides, a TLR3 agonist, mono-phosphoryl-lipid A (MPL), MPL-trehalose 6,6′-dimycolate (MPL-TDM), and anti-CD40.

The invention further provides for the use of a composition comprising purified HspE7 and an immune stimulant selected from the group consisting of CpG containing oligonucleotides, a TLR3 agonist, mono-phosphoryl-lipid A (MPL), MPL-trehalose 6,6′-dimycolate (MPL-TDM), and anti-CD40 for the treatment of a condition related to an HPV infection.

The invention further provides for a composition comprising purified HspE7 and an immune stimulant selected from the group consisting of CpG containing oligonucleotides, a TLR3 agonist, mono-phosphoryl-lipid A (MPL), MPL-trehalose 6,6′-dimycolate (MPL-TDM), and anti-CD40 for use in the manufacture of a medicament for the treatment of a condition related to an HPV infection.

A condition related to an HPV infection may be a chronic infection, or a pathology associated with an HPV infection; the HPV infection may include an HPV of one or more types 1-5, 6, 7, 8, 9 10, 11, 12, 13, 14, 15, 16, 17, 18, 19, 20 31, 32, 33, 35, 36, 38, 39, 41, 47, 48, 50 or 75-77.

Examples are provided herein (e.g. Example 13) illustrating the use of compositions according to some embodiments of the invention in treating a subject having an HPV infection, and that such compositions are safe, tolerated and immunogenic in humans. The increased biological activity of the immunizing antigen in human subjects is illustrated, and examples of indicators of biological activity (antibody response, CD4-positive and CD8-positive T cell responses) to the immunizing antigen are disclosed.

In some embodiments of the invention, a kit comprising purified HspE7, an immune stimulant, and instruction for use is provided. The immune stimulant may include CpG-containing oligonucleotides, TLR3 agonists such as PolyI:C or polyICLC, mono-phosphoryl-lipid A (MPL), MPL-trehalose 6,6′-dimycolate (MPL-TDM), and anti-CD40, including but not limited to polyIC nucleic acids having any of the nucleosides, internucleoside linkages and compositions described herein. The kit may provide single-dose formulations of purified HspE7 and an immune stimulant, pre-packaged in a single-use device, for example a patch, implant or syringe. Alternately, the kit may provide a multi-dose formulation that may be divided in to single dose units at a pharmacy or at the point of administration by a physician or other suitable person.

The present invention is using conventional techniques of molecular biology, microbiology, virology, recombinant DNA technology, peptide synthesis in solution, solid phase peptide synthesis, and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. Oligonucleotide Synthesis: A Practical Approach (M. J. Gait, ed., 1984) IRL Press, Oxford, including Gait, pp. 1-22; Atkinson et al., pp. 35-81; Sproat et al., pp. 83-115; and Wu et al., pp. 135-151;
3. Animal Cell Culture: Practical Approach, Third Edition (John R. W. Masters, ed., 2000), ISBN 0199637970;
4. Immobilized Cells and Enzymes: A Practical Approach (1986) IRL Press, Oxford, whole of text;
5. J. F. Ramalho Ortigao, “The Chemistry of Peptide Synthesis” In: Knowledge database of Access to Virtual Laboratory website (Interactive, Germany);
6. Barany, G. and Merrifield, R. B. (1979) in The Peptides (Gross, E. and Meienhofer, J. eds.), vol. 2, pp. 1-284, Academic Press, New York;
7. Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg;
8. Handbook of Experimental Immunology, Vols. I-IV, D. M. Weir and C. C. Blackwell, eds., 1986, Blackwell Scientific Publications.

### EXAMPLES

Immune Monitoring Methods and Materials:

### Peptides

Sets of fifteen-mer peptides with an 11 amino acid overlap, and spanning the entire open reading frames of HPV-16 E7 and HPV-6 E7 were purchased from JPT Peptide Technologies (Berlin, Germany). Peptides were purified by HPLC to >90%. Individual peptides from each HPV type (22 peptides) were dissolved in DMSO and pooled for a working concentration of 1 mg per peptide/mL. Individual peptides are listed in Table 1

TABLE 1
Peptides derived from the amino
acid sequence of HPV type 16 E6
(SEQ ID NO: 23) and E7 (SEQ ID NO: 24)

| SEQ ID NO: | Sequence | peptide # |
|---|---|---|
| 1 | mhgdtptlheymldl | 1 |
| 2 | tptlheymldlqpet | 5 |
| 3 | heymldlqpettdly | 9 |
| 4 | ldlqpettdlycyeq | 13 |
| 5 | pettdlycyeqlnds | 17 |
| 6 | dlycyeqlndsseee | 21 |
| 7 | yeqlndsseeedeid | 25 |
| 8 | ndsseeedeidgpag | 29 |
| 9 | eeedeidgpagqaep | 33 |
| 10 | eidgpagqaepdrah | 37 |
| 11 | pagqaepdrahyniv | 41 |
| 12 | aepdrahynivtfcc | 45 |
| 13 | rahynivtfcckcds | 49 |
| 14 | nivtfcckcdstlrl | 53 |
| 15 | fcckcdstlrlcvqs | 57 |
| 16 | cdstlrlcvqsthvd | 61 |
| 17 | lrlcvqsthvdirtl | 65 |
| 18 | vqsthvdirtledll | 69 |
| 19 | hvdirtledllmgtl | 73 |
| 20 | rtledllmgtlgivc | 77 |
| 21 | dllmgtlgivcpics | 81 |
| 22 | gtlgivcpicsqkp | 85 |

CEF peptide pool (catalog No. 3615-1) was purchased from Mabtech (Cincinnati, Ohio). The CEF pool contains 32 8-12-mer peptides representing immunodominant CD8+ T cell epitopes from human cytomegalovirus, Epstein Barr virus and influenza virus that are recognized by T cells of approximately 70% of the U.S. population.

### Cryopreservation of PBMC and Serum Samples

Whole blood was drawn by venipuncture into yellow top ACD-A-containing blood tubes and/or a red top serum tube at 4 time points: baseline, 1 week, 5 weeks, 9 weeks, and 12 weeks following initiation of therapy. Unprocessed venipuncture tubes were shipped overnight at ambient temperature to a central processing lab where immunomonitoring protocols were carried out. Blood tubes not received within 48 h of blood collection were discarded. PBMC were isolated by standard gradient centrifugation over human Lymphocyte Separation Medium (Mediatech, Herndon, Va.) using Leucosep tubes (Greiner Bio-One, Monroe, N.C.). PBMC at the ficoll interface were collected, washed, and counted using 0.4% Trypan Blue solution (Mediatech). Cells were cryopreserved in 10% DMSO/40% FBS/50% RPMI1640 at a concentration of 10-30×10⁶ PBMC/mL by placing vials into a controlled rate freezing container (Nalgene, Rochester, N.Y.) overnight at −80° C. Vials were transferred to liquid nitrogen the following day for long-term storage. For serum collection, clotted blood was centrifuged at 2000 rpm for. 10 min at room temperature. Serum was collected, aliquoted, and stored at −80° C. for long-term storage. For all assays, cells or serum from one subject at each time point were batched and run side by side on the same day.

### IFNγ ELISPOT

Cryopreserved PBMC were quickly thawed in a 37° C. water bath, transferred to a sterile tube, then washed twice with RPMI-1640 supplemented with 10% human AB serum (Sigma, St. Louis, Mo.), 2 mM L-Glutamine, 50 μg/ml kanamycin, 1 mM sodium pyruvate, 2 mM non-essential amino acids, and 50 μM 2-mercaptoethanol (i.e. complete medium). Cells were “rested” in 37° C., 5% CO₂ incubator overnight in 5 mL complete medium supplemented with 10 U/mL Benzonase nuclease (EMD Biosciences (Gibbstown, N.J.) to remove dead and/or apoptotic cells. After resting, cells were harvested, washed and counted on a hemacytometer using trypan blue to exclude dead cells. Cells were plated at a density of 2×10⁶ viable cells per well in a 24-well plate in 2 mL complete medium in the presence or absence of 5 μg/mL of HPV16 E7 peptides combined in pools. As a positive control, PBMC were cultured with 2.5 μg/mL of the CEF peptide pool. After 4 days of incubation, cells were harvested, washed, counted and plated in four replicate wells of a sterile PVDF-backed 96-well microtiter plate (Millipore, Billerica, Mass.) pre-coated with interferon gamma (IFNγ) capture antibody (1-D1K, Mabtech). Cells were plated at 100,000 cells/well for medium control wells and peptide pool wells and 50,000 cells/well for PHA positive control wells. PHA-L (Sigma, St. Louis, Mo.) was added at a final concentration of 5 μg/ml. Plates were incubated for 18 h in a humidified 37° C., 5% CO₂ incubator. After 18 h, cells were removed by washing and plates incubated with biotinylated detection IFNγ antibody (7-B6-1, Mabtech, 1 μg/ml) for 2 h at room temperature. Following washing, wells were incubated with streptavidin-peroxidase (Sigma, diluted 1/4000) for 1 h at room temperature. Spots were developed with AEC (3-amino-9-ethyl-carbazole) substrate (Sigma) dissolved in 0.05 M sodium acetate buffer. Reaction was stopped by washing extensively in deionized water, and plates were left to dry overnight at room temperature. Spots were imaged and counted using the automated computer-assisted video-imaging KS ELISPOT analysis system (Carl Zeiss, Thornwood, N.Y.). The number of peptide-specific spots was calculated by subtracting the mean number of spots from medium control wells from the mean number of spots from experimental wells.

### Proliferation Assay

Cryopreserved PBMC were quickly thawed in a 37° C. water bath, transferred to a sterile tube, then washed twice with RPMI-1640 supplemented with 10% human AB serum (Sigma, St. Louis, Mo.), 2 mM L-Glutamine, 50 μg/ml kanamycin, 1 mM sodium pyruvate, 2 mM non-essential amino acids, and 50 μM 2-mercaptoethanol (i.e. complete medium). Cells were “rested” in 37° C., 5% CO₂ incubator overnight in 5 mL complete medium supplemented with 10 U/mL Benzonase nuclease (EMD Biosciences (Gibbstown, N.J.). After resting, cells were harvested, washed and counted on a hemacytometer using trypan blue to exclude dead cells. Cells were plated in six replicate wells at a density of 1.5×10⁶ cells per well in a 96-well round-bottom plate (Costar) in 0.2 mL complete medium in the presence or absence of 10 μg/mL of HPV16 E7 or HPV6 E7 peptides combined in pools. As a positive control, PBMC were cultured with 5 μg/mL PHA-L added on day 4 of culture. On day 6 of culture, cells were pulsed with 0.5 μCi [³H]thymidine per well and incubated for an additional 18 h. Cells were harvested onto a 96-well Unifilter plate and counted using a TopCount Scintillation Counter (Perkin Elmer, Foster City, Calif.). Thymidine uptake in counts per minute (cpm) was determined by averaging replicate wells.

### HspE7 ELISA Assay

Serum HspE7 antibodies were assayed using a custom developed ELISA assay. Briefly, 96-well Maxisorp polystyrene plates (Nunc, Rochester, N.Y.) were coated with 1 μg of HspE7 in PBS or PBS alone and incubated overnight at 4° C. Plates were blocked with Starting Block blocking solution (Pierce). Thawed serum samples were added to plates in triplicate wells in five-fold dilutions starting with a ten-fold dilution of serum in Starting Block supplemented with 0.05% Tween-20 and incubated for 2 h. Following washing, goat anti-human Ig-peroxidase (Pierce, 1:5000 dilution) was added to wells for 1 h. Bound antibody was detected by the addition of o-phenylenediamine substrate. Reactions were stopped after 5 min with 2N H₂SO₄ and absorbance values read on a Chameleon V plate reader at 490 nm. Absorbance values from PBS-coated wells were subtracted from absorbance values from HspE7-coated wells. Cutoff values for a positive reaction in experimental wells were determined by subtracting the average absorbance readings from no serum control wells plus 3×SD. HspE7 antibody titer is reported as the inverse of the highest dilution giving a positive response above the cutoff value.

The present invention will be further illustrated in the following examples.

### Example 1

### HspE7 Preparation

The Hsp65-E7 fusion (HspE7) was obtained as described in WO99/07860 (which is incorporated herein by reference). HspE7 is a fusion protein comprising the complete HPV16 E7-coding region inserted at the carboxy-terminal end of the Hsp65 gene (pET65H). This HspE7 is referred to as Process A HspE7, and is available from Nventa Biopharmaceuticals Corporation by request.

Prior to use, HspE7 is purified to greater than 95% purity. A seed culture of HspE7 expressing E. coli was used to inoculate 250 L of fermentation medium. During the fermentation process yeast extract and glucose were added as feed, and pure oxygen was sparked into the fermentation vessel, to supply sufficient aeration. Expression of HspE7 was induced by the addition of IPTG (isopropyl-β-D-thiogalactopyranoside). The content of the fermenter was then cooled to <20° C. and the cell paste harvested by centrifugation. The cell paste was re-suspended in buffer containing urea and sulfitolysis reagents. The sulfitolysis reagents converted the sulfhydryls-groups in HspE7 into S-sulfocysteine. The HspE7-solution was clarified by precipitation with PEI (polyethyleneimine), followed by a precipitation of the product at its pI. HspE7 was then purified to homogeneity using a series of cation and anion-exchange chromatography steps, and the modified sulfhydryls were reduced with DTT (dithiothreitol). Finally, HspE7 underwent an ultrafiltration and diafiltration into Histidine/mannitol buffer, and stored at −70° C. The purified form of HspE7 is termed Process L HspE7. The purity of HspE7 was determined via gel electrophoresis.

As outlined below, the highly purified, Process L HspE7 was observed to lose biological activity when compared to the less pure (Process A HspE7) product. The less pure HspE7 product (Process A HspE7) exhibited biological activity as disclosed in the WO99/07860.

### Example 2

### Determination Biological Activity of HspE7 Preparations

### Antigen-Specific Stimulation of Splenocyte Production of INF-Gamma: ELISPOT Assay

Augmentation of the ability of HspE7 to induce E7-specific CD8-positive T lymphocytes (IFN-gamma producing cells) was determined in the presence of E7 peptide by ELISPOT (Asai, T., et. al., 2000, Clin. Diagn. Lab. Immunol. 7(2):145-154) as follows: Mice were immunized with HspE7, with or without the addition of adjuvants, subcutaneously in the scruff of the neck in a total volume of 200 ul. Five to seven days later the mice were sacrificed, their spleens removed and processed to a single cell suspension. Cells were plated in complete RPMI onto Millipore filter plates previously coated with anti-mouse IFN-gamma antibodies. The plates were incubated at 37° C. for 20 hours. The cells were washed off and IFN-gamma spots were detected by incubation of the plates with a biotinylated secondary anti-mouse IFN-gamma antibody. Spots were visualized with Vectastain ABC Elite kit and AEC substrate. Spots were counted on a Zeiss Automated ELISPOT counter.

### Tumor Regression Assay

Tumor regression was determined using an assay comprising the tumor cell line TC-1.K, a lung epithelial tumor stably transfected with HPV16 E6 and E7 oncogenes. TC-1.K cells were implanted in mice followed by a test sample injection 7 days later and regular tumor palpation thereafter. The assay involved seeding TC.1K tumor cells for culture and expanding cell numbers prior to implanting within C57BL/6 mice, 7-14 weeks of age, essentially as described by Chu N. R., et. Al. (Chu N. R. et. al., 2000, Clin Exp Immunol 121 (2):216-225). After 7 days post tumor implantation, tumor-bearing mice were treated with test and control samples. Typically groups of 180 mice are divided into 6 equal groups, and each group is injected with either a control (vehicle only), or 50, 100, 200, 400 or 800 μg of HspE7 Reference Sample. Mice were palpitated for tumor at 14, 28 and 49 days.

As shown in FIG. 1, the anti-tumor activity of Process A HspE7 is greater than that of Process L HspE7, with lower doses achieving the same or reduced tumor incidence when compared to a similar dose of Process L HspE7. For this assay, mice bearing established TC-1 tumors were injected subcutaneously in the scruff of the neck with graded doses of HspE7 produced by either process A or process L (n=30/grp/dose) and followed for tumor growth for 49 days.

### Example 3

### Effect of the TLR9 Agonist CpG on HspE7

Augmentation of the ability of HspE7 to induce E7-specific CD8-positive T lymphocytes was determined in the presence of CpG oligonucleotides (a TLR9 agonist). Naïve C57Bl/6 mice were injected subcutaneously as described in Example 2, with either HspE7 alone, produced by two different purification processes (400 ug Process A HspE7 or 400 ug of Process L HspE7), or HspE7 (either 400 ug Process A HspE7 or 400 ug Process L HspE7) plus 30 ug of CpG (TCC ATG ACG TTC CTG ATG CT; SEQ ID NO:1; available from Invitrogen, comprising a phosphorothioate backbone and is designated: ZOO FZE FOE ZZO OZE FZE OT). Five days later, spleens were removed from the mice and the number of E7-specific splenocytes was measured by ELISPOT using E7 specific class I MHC binding peptide E7₄₉₋₅₇ (RAHYNIVTF; Dalton Chemical Laboratories), or the control peptide HBCAg₉₃₋₁₀₀ (MGLKFRQL; Dalton Chemical Laboratories) as recall antigens.

The results shown in FIG. 2 indicates that purified HspE7 (Process L HspE7) exhibits minimal induction of E7-specific CD8-positive T lymphocytes. Process A HspE7, exhibits a greater induction of—E7-specific CD8-positive T lymphocytes. However, the induction of both Process L HspE7 and Process A HspE7 is enhanced from 3 to 100 fold in the presence of the TLR9 agonist CpG (Process A HspE7+CpG or Process L HspE7+CpG).

Several CpG-containing oligonucleotides with the optimal murine class B type core sequence (GACGTT), including 1668, were shown to be highly active in augmenting the activity of HspE7 in the ELISPOT assay and the TC-1 tumor regression assay (data not shown). Similarly a CpG class C type oligonucleotide (2395) was found to be highly active. However a class A type CpG-containing oligonucleotide was found to be far less effective in augmenting the activity of HspE7 in the ELISPOT assay (see Vollmer J., et al. 2004 Eur. J. Immunol. 34:251-262).

These data demonstrate that purified HspE7 is biologically active, and that the biological activity of HspE7 (either Process A or Process L HspE7) may be increased by adding the immune stimulant, CpG.

### Example 4

### Effect of Additional TLR Agonists on HspE7

The ability of alternate TLR agonists to augment HspE7 (Process L HspE7) induction of E7-specific CD8-positive T lymphocytes was determined using the TLR3 agonist, PolyI:C (Sigma Cat #P1913), and the TLR2 agonist PAM3CysSK4 (Invivogen Cat #TLR1-pms), and the TLR9 agonist CpG (see Example 3).

Mice were co-injected subcutaneously with a mixture of HspE7 plus TLR-agonist. For this study 50 ug of Process L HspE7 was co-injected along with 10 ug CpG, 20 ug of Pam3CysSK4 or 100 ug PolyI:C. Five days later, spleens were removed from the mice and the number of E7-specific splenocytes was measured by ELISPOT (as outlined in Example 3) using the E7 specific class I MHC binding peptide E7₄₉₋₅₇, or the control peptide HBCAg₉₃₋₁₀₀ as recall antigens. The results are shown in FIG. 3.

As can be seen in FIG. 3, the co-injection of HspE7 and CpG, results in a significant augmentation of E7-specific CD8-positive T lymphocytes. A similar increase is also observed with co-administration of the TLR3 agonist PolyI:C. However, the TLR2 agonist Pam3CysSK4 only resulted in a negligible augmentation of IFN-gamma producing cells.

These results indicate that CpG and PolyI:C, but not Pam3CysSK4, are effective in augmenting purified HspE7 (Process L HspE7), and that not all adjuvants are effective in augmenting biological activity of purified HspE7. Additional experiments (FIGS. 4 and 5) demonstrate that admixing purified HspE7 with PolyICLC (Oncovir, 3203 Cleveland Ave NW, Washington D.C.) or with MPL-trehalose 6,6′-dimycolate (MPL-TDM; from Ribi ImminoChem Research Inc.; also see Oiso R., et al., Microb Pathog. 2005 July-August; 39(1-2):35-43) also were effective in augmenting the immunological activity of purified HspE7 (i.e. greater than 95% pure). In FIG. 5, it is shown that an augmentation of the immunological activity of HspE7 can be detected over a 1000 fold amount of immuno-stimulant, with augmentation in activity observed from 0.1 ug of PolyICLC to 100 ug polyICLC.

### Example 5

### Anti-Tumor Activity of HspE7

The effect of purified HspE7 preparations on anti-tumor activity was examined using the method outlined in Example 3. The data show that combining purified HspE7 with CpG or PolyI:C significantly increases the anti-tumor efficacy compared to purified HspE7 (Process L HspE7) alone.

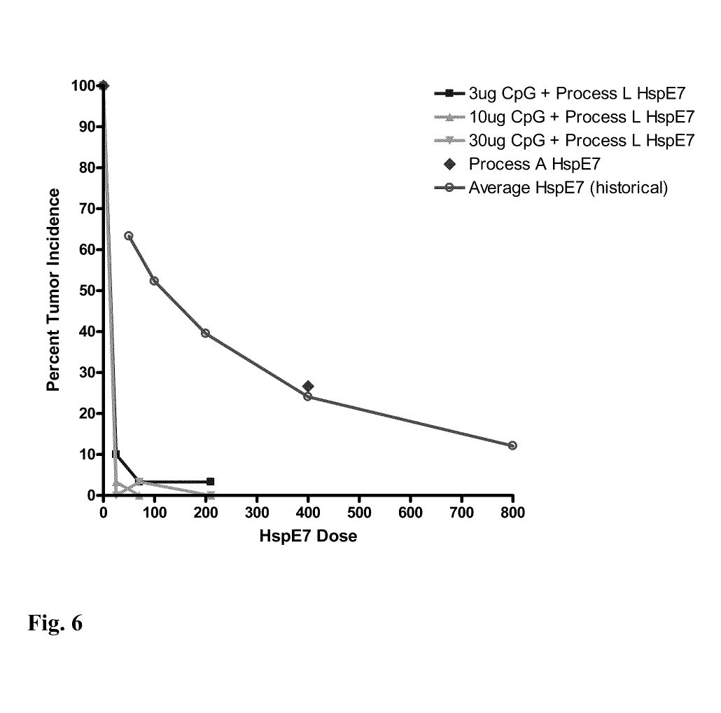

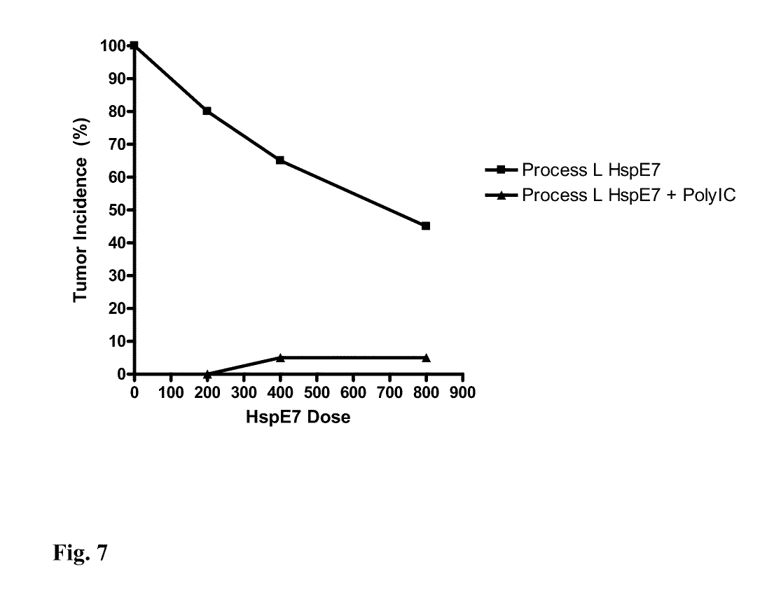

Mice were injected in the flank with 6×10⁴ TC-1 tumor cells. On day 7, mice bearing established TC-1 tumors were injected subcutaneously in the scruff of the neck with either diluent, purified HspE7 alone (prepared as in Example 1; Process L; HspE7 of less than 95% purity (FIG. 6) or graded doses of purified HspE7 mixed with different doses of CpG (n=30/grp), or PolyI:C (n=20/grp; FIG. 7). Mice were followed for tumor growth for an additional 42 days. Mice free of tumor 49 days post tumor implantation were considered to be tumor free. One hundred percent of mice injected only with diluent had tumors on day 49. Previous studies had demonstrated that CpG alone, or PolyI:C alone has no effect on tumor growth (data not shown).

The results for HspE7 co-administered with CpG are shown in FIG. 6, and for HspE7 co-administered with PolyI:C are shown in FIG. 7.

With reference to FIG. 6, the administration of HspE7 of less than 95% purity reduced tumor activity over a dose ranges of 50 to 800 ug (HspE7 and Average HspE7 historical), with about 15% tumor incidence being observed at high (800 ug) of HspE7 (“historical”). However, co-injection of purified HspE7 with CpG resulted in a dramatic decrease in tumor incidence with doses of about 25 to about 200 ug HspE7 resulting in less than 5% tumor incidence. Approximately 27 percent of mice treated with 400 ug of Process B HspE7 had tumors on day 49 as would be predicted from historical data. However, 90% of the mice injected with as little as 25 ug of Process L HspE7 mixed with 3 ug of CpG had complete tumor clearance.

With reference to FIG. 7, it can be seen that the administration of purified HspE7 (greater than 95% purity; Process L HspE7) was not as potent as 95% purity HspE7 in reducing tumor activity over a dose range of up to 800 ug (HspE7), with about 50% tumor incidence being observed at high (800 ug) of HspE7. However, co-injection of purified HspE7 with 100 ug polyI:C resulted in a dramatic decrease in tumor incidence with doses of about 200 ug HspE7 resulting in less than 5% tumor incidence.

These data demonstrate that purified HspE7 exhibits anti-tumor activity which is increased from about 5 to about 80 fold when administered with a TLR9 agonist such as CpG, or the TLR3 agonist PolyI:C.

### Example 6

### Effect of Traditional Adjuvants on HspE7 Activity

The ability of traditional adjuvants such as alum, or Freunds Incomplete Adjuvant (IFA) to augment purified HspE7 induction of E7-specific CD8-positive T lymphocytes was determined.

Mice were injected subcutaneously with purified HspE7 (400 ug Process L HspE7; Process L or co-injected with purified HspE7 (400 ug) along with 30 ug CpG, mixed 1:1 with Alum (Pierce), or mixed 1:1 with IFA (Bacto), or along with alum+30 ug CpG, or along with IFA+30 ug CpG. Five days later, spleens were removed from the mice and the number of E7-specific splenocytes was measured by ELISPOT using the E7 specific class I MHC binding peptide E7 (49-57) (16.E7.49-57.Db), or the control peptide HBCAg (93-100) as recall antigens. The results are shown in FIG. 8.

In agreement with the results presented in the Examples 3 and 4, injection of purified HspE7 alone (Process L HspE7) did not augment the production of IFN-gamma by splenocytes, but co-injection of HspE7 and CpG (Process L HspE7+CpG), resulted in significant (over 10 fold) augmentation of E7-specific CD8-positive T lymphocytes (FIG. 8). However, co-injection of purified HspE7 with IFA (Process L HspE7+IFA) or with alum (Process L HspE7+Alum), did not result in any appreciable augmentation of the stimulation of E7-specific T lymphocytes, matched the effect of administering purified HspE7 alone.

Co-administration of purified HspE7 along with CpG and either alum (Process L HspE7+Alum+Cpg) or IFA (Process L HspE7+IFA+Cpg), resulted in augmentation of the stimulation of E7-specific T lymphocytes similar to the augmentation observed with HspE7 co-injected with CpG (Process L HspE7+CpG). This data demonstrates that IFA and Alum are neutral, neither inhibiting or stimulating HspE7, as CpG has a similar effect on augmenting the activity of HspE7 regardless of whether or not Alum or IFA are present.

These data show that induction of E7-specific, CD8-positive T lymphocytes is not augmented by mixing purified HspE7 with either of the well known adjuvants alum or Freunds Incomplete Adjuvant (IFA). These results further demonstrate that not all adjuvants, including alum and Freunds Incomplete Adjuvant, are effective in augmenting biological activity of purified HspE7.

### Example 7

### Effects of Additional TLR Agonists on HspE7 Activity

In this example, the effect of imiquimod, LPS, Pam3CysSK4, or anti CD40 to augment purified HspE7 induction of E7-specific CD8-positive T lymphocytes was determined.

Mice were injected subcutaneously with a mixture of purified HspE7 (400 ug Process L HspE7; process L), or purified HspE7 (400 ug) along with 100 ug imiquimod (Invivogen #TLRL-IMQ), 30 ug LPS (Sigma), 25 ug Pam3CysSK4 (Invivogen Cat #TLR1-pms) 25 ug anti-CD40 (R&D Systems; clone number 1C10), or 30 ug CpG. Five days later, spleens were removed from the mice and the number of E7-specific splenocytes was measured by ELISPOT using the E7 specific class I MHC binding peptide E7₄₉₋₅₇. The results are shown in FIG. 9.

Co-administration of purified HspE7 with imiquimod (a TLR7 agonist), PAM3CysSK4 (a TLR2 agonist) or LPS (a TLR4 agonist) only weakly augmented the ability of purified HspE7 to induce E7-specific T lymphocytes (FIG. 9). However, stimulation in the generation of E7-specific CD8-positive T lymphocytes was observed by adding anti-CD40 or CpG to HspE7.

These results further demonstrate that not all TLR agonists are effective at augmenting the generation of E7-specific CD8-positive T lymphocytes as only a modest increase in the number of IFN-gamma secreting cells was observed by adding imiquimod (TLR7 agonist), PAM3CysSK4 (TLR2 agonist) and LPS (TLR4 agonist) to HspE7.

### Example 8

### Daily Injection Scheme

HspE7 and polyICLC were used to assess the utility of a daily injection regime to elicit CD8+ T-cell responses in mice. C57Bl/6 mice (2 per group) were immunized with HspE7 (100 ug) and polyICLC (10 ug) at daily intervals, once per day up to a maximum of 4 days. 7 days after the first exposure to antigen, all animals were euthanized and their splenocytes taken for analysis. IFN-gamma ELISPOT was used to assess the class 1-restricted CD8+ T-cell response upon stimulation with 16E7.49-57.Db peptide.

Groups given multiple injections of HspE7+polyICLC displayed a measurable increase in the frequency of the response as compared to the group given a single injection.

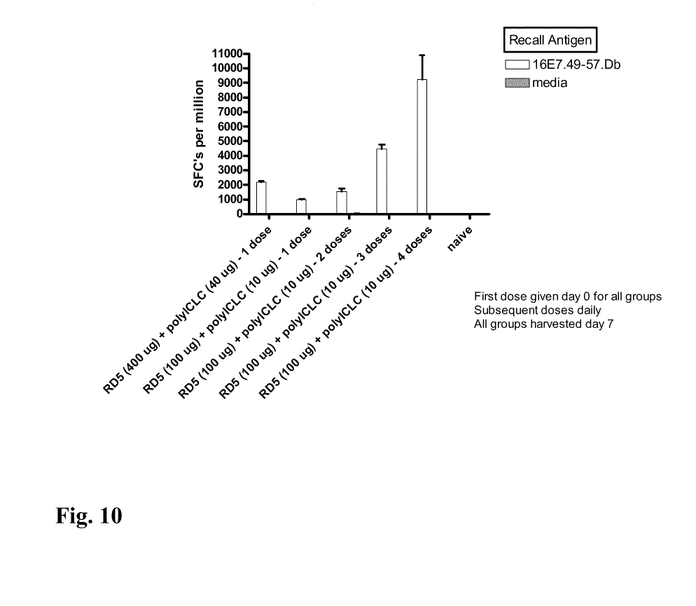

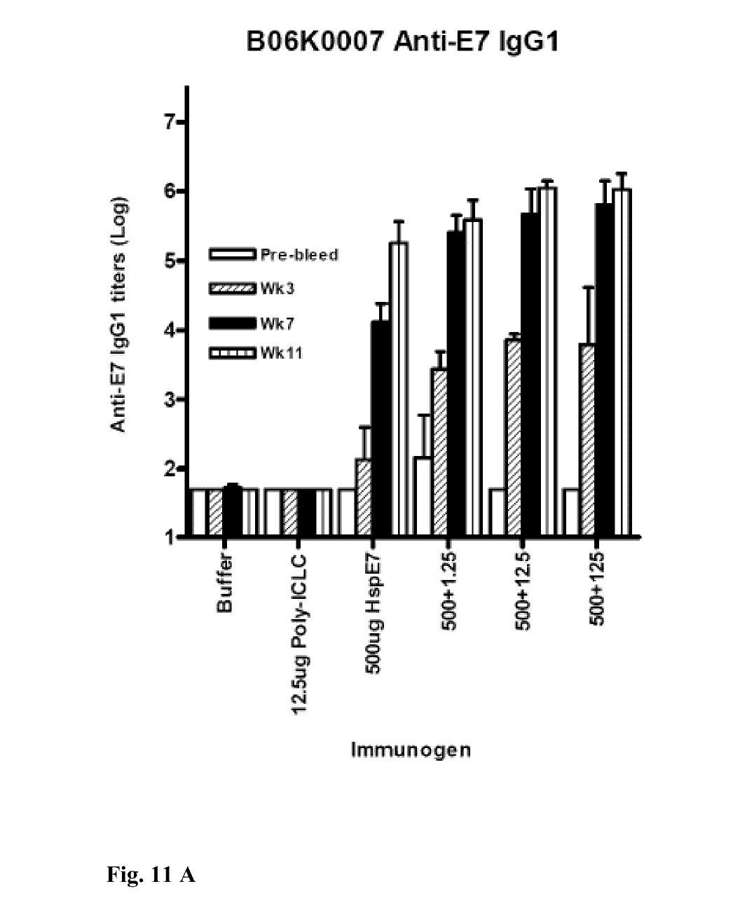

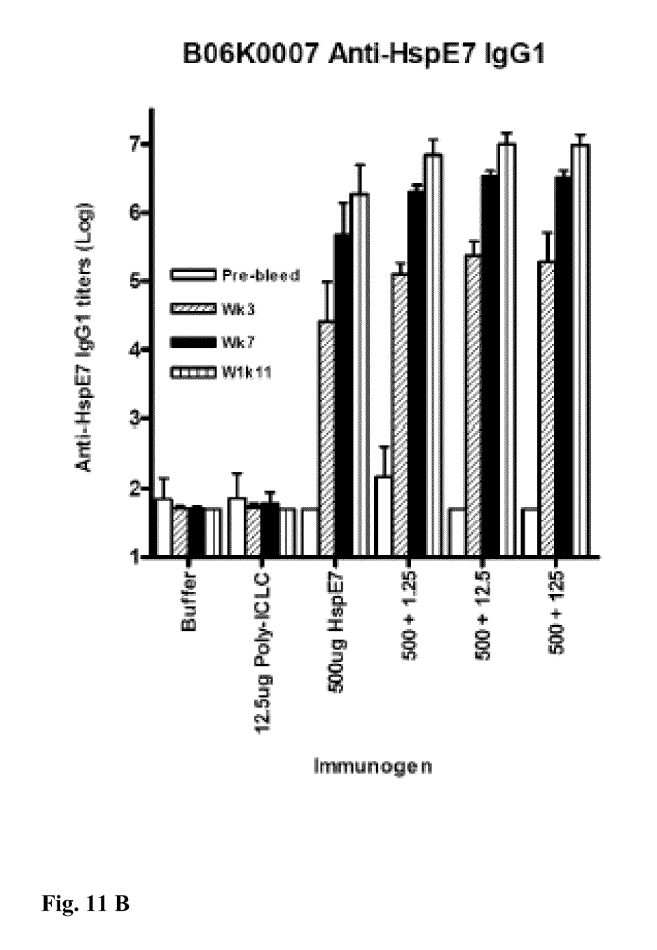

Increasing the number of daily injections correlates with an increase in the frequency of the response. The group given the largest number of daily injections showed the largest increase in the frequency of the response (FIG. 10).

The use of a daily injection strategy should provide utility in eliciting increased CD8+ T-cell responses using polyICLC in combination with other CoVal™ antigens, or in combination with non-CoVal™ antigens. Additionally, this strategy may result in a larger CD8+ memory pool that may have an increased ability to boost the subsequent immune response upon re-challenge at weekly or biweekly intervals.

### Example 9

### Humoral Response to Immunization with HspE7 Plus Poly-ICLC

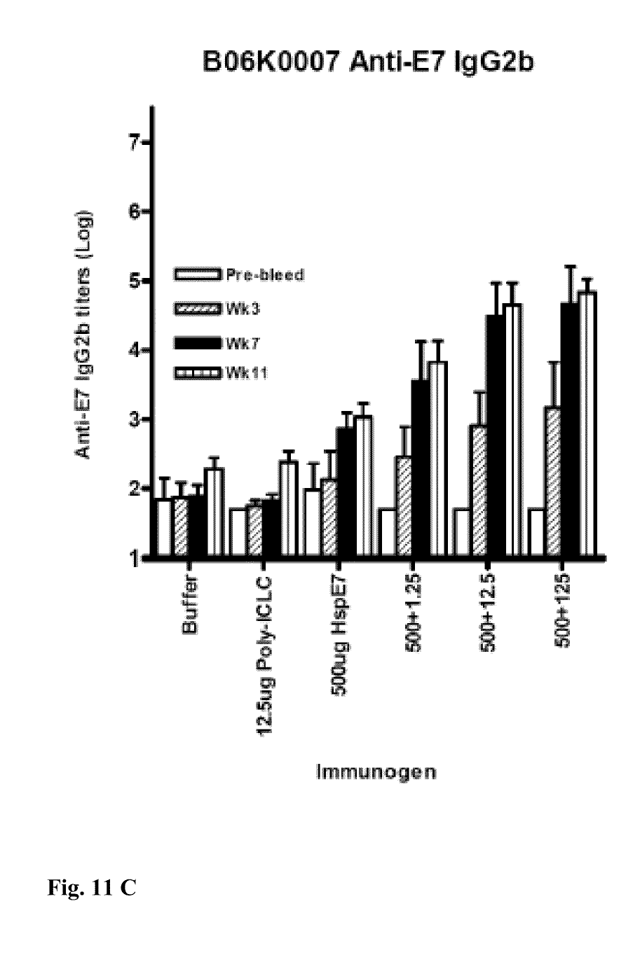

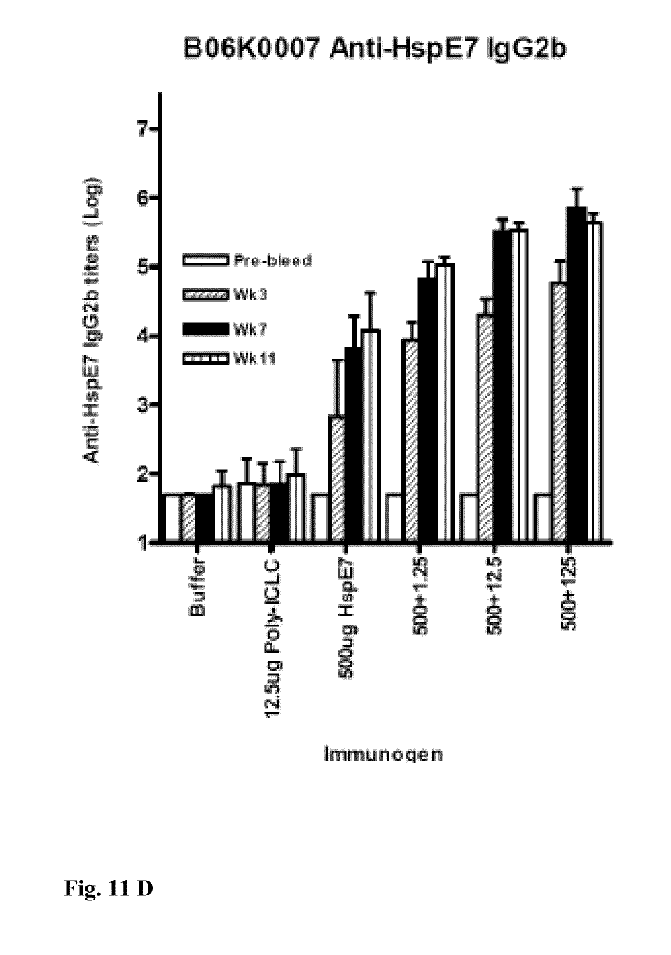

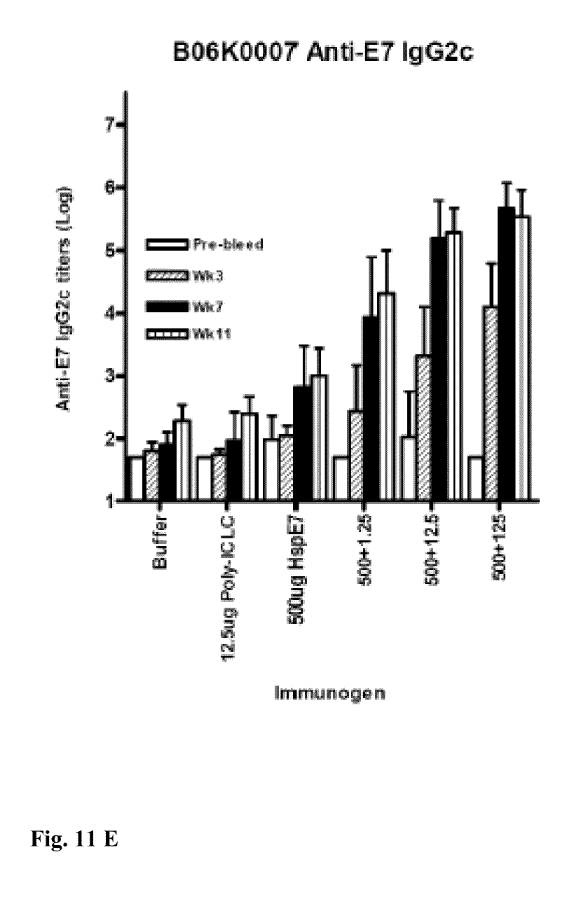

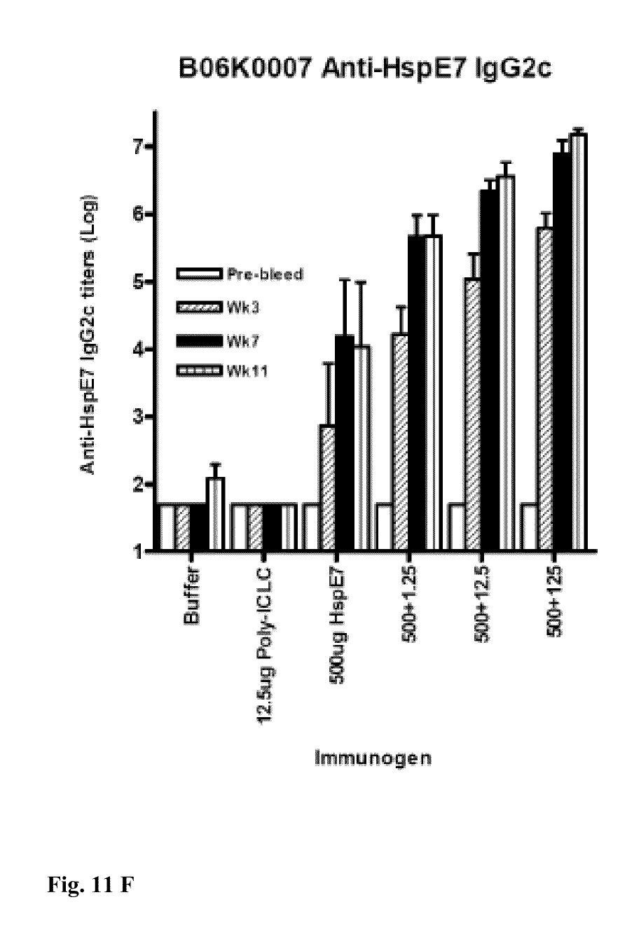

Poly-ICLC has been demonstrated to augment both cellular and humoral immune responses to antigens. To investigate the effect of co-immunization with HspE7 plus Poly-ICLC on humoral immunity, groups of C57Bl/6 female mice (n=5/group) were immunized 2 times at monthly intervals (day 1, 28). Groups of mice were immunized with buffer, 500 μg HspE7, 12.5 μg Poly-ICLC, 500 μg HspE7+1.25 μg Poly-ICLC, 500 μg HspE7+12.5 μg Poly-ICLC or 500 μg HspE7+125 μg Poly-ICLC. Blood samples were taken for analysis of serum antibodies 7 days prior to dosing (d-7, baseline) and at days 21, 49 and 77). Sera from individual mice were tested for the presence of antibodies (IgG1, IgG2b and IgG2c) to E7 and HspE7 by standard ELISA. Briefly, 96 well plates were coated overnight with E7 or HspE7, washed and blocked with a 1.5% BSA solution. Sera was added to individual wells in 2 fold serial dilutions starting at a 1 in 50 dilution of sera in BSA solution. Following washing, bound IgG1 (FIGS. 11A, B), IgG2b (FIGS. 11C, D) or IgG2c (FIG. 11E, F) antibodies to HspE7 (FIGS. 11B, D, F) or E7 (FIGS. 11A, C, E) antigens were detected by incubation with biotin conjugated antibodies against the appropriate immunoglobin isotype. The plates were then washed and incubated with streptavidin conjugated horseradish peroxidase. Color development was done using tetramethylbenzidine (TMB) substrate and the colored product was read at 450 nm in an automated ELISA plate reader. Data in FIG. 11 are expressed as the highest dilution of sera that gave an absorbance greater than the assay plate background (defined as 0.2 OD units).

Immunization with HspE7 alone, produced significant antibody responses to both HspE7 and E7, with the anti-HspE7 response being pronounced following a single injection, while the anti-E7 response was weak following a single injection and developed more fully following two immunizations. In both cases, the isotype of the antibodies produced was predominantly IgG1 (FIGS. 11A, B), indicating a Th2 shifted humoral response. Immunization with Poly-ICLC alone did not produce an antibody response to either E7 or HspE7. Immunization with HspE7 plus Poly-ICLC resulted in stronger and more rapidly developing antibody responses. This was most pronounced in the case of E7 where the immune response was notable following a single injection when Poly-ICLC was co-immunized with HspE7. Further, there was a significantly greater Th1 humoral response, resulting in an increased proportion of IgG2b&c (FIGS. 11C-F) antibody isotypes being produced. This response was dose dependant as it was more marked at higher doses of Poly-ICLC. The increased Th-1 shift of the immune response when HspE7 and Poly-ICLC are co-injected is consistent with the increased magnitude of IFN-gamma producing CD8-positive T lymphocytes observed by ELISPOT.

### Example 10

### Dose Range of HspE7 with PolyICLC

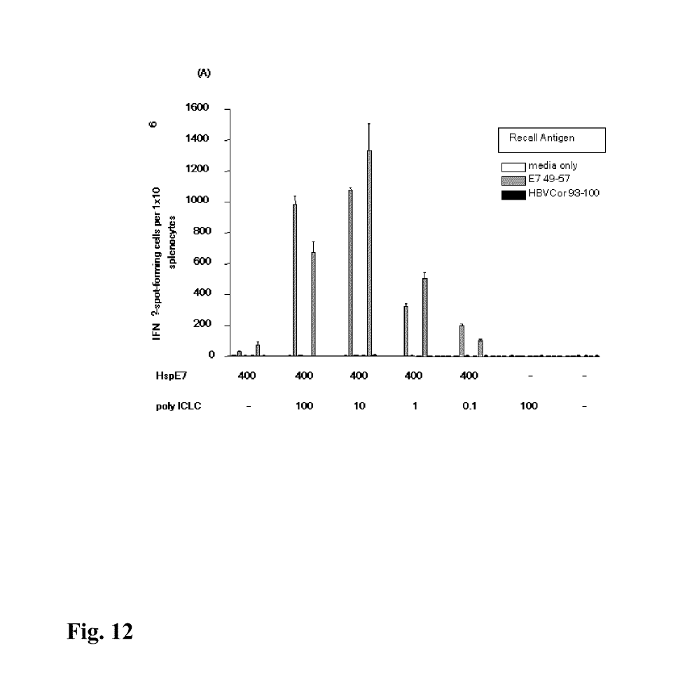

A dose range over which a TLR3 agonist was able to promote the cross-priming of E7-specific CD8 T cells when co-delivered in combination with HspE7 was explored. As shown in FIG. 12, we observed that immunization of mice with HspE7 plus the TLR3 agonist polyICLC was highly efficient at eliciting E7₄₉₋₅₇-specific T cells as measured by IFN-gamma ELISPOT. The number of E7₄₉₋₅₇-specific cells elicited was dependent upon the dose of polyICLC adjuvant used, however even very low doses (0.1 ug polyICLC) were able to augment the cross-priming of HspE7.

### Example 11

### Regression of Large, Established Tumors with Consecutive Daily Doses of HspE7 Plus PolyICLC

The E7-expressing TC-1 tumor cell line is an aggressive, rapidly growing tumor model that is widely used to assess the effectiveness of E7-directed vaccination strategies. Generally, mice are implanted with between 10⁵ and 10⁶ cells of a TC-1 tumor cell line and are treated with the agent of interest 7 to 14 days later, once the tumor is palpable. In this tumor model, there is a therapeutic window after which time immunological intervention is no longer useful because the tumor grows so fast than clonal expansion of antigen-specific T cells cannot overcome the tumor before the tumor burden becomes overwhelming.

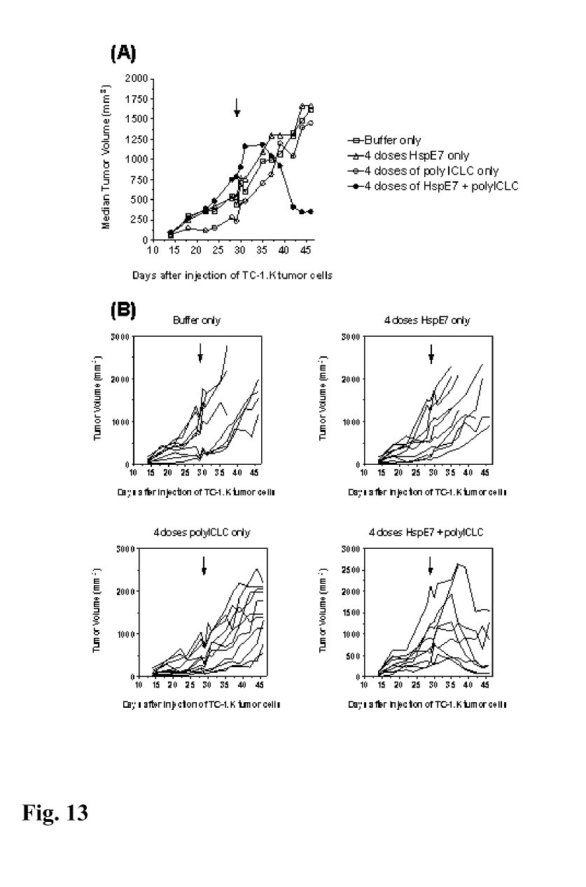

The TC-1 tumor model system used in these experiments allowed for a more advanced tumor to develop. As illustrated in FIG. 13, TC-1 tumors were allowed to grow in vivo for 28 days prior to treatment, rather than the conventional 7 to 14 days. Although there was a large range in the average tumor size at this time point, all animals had palpable tumors and some animals had tumors with a volume exceeding 2000 mm³. Remarkably, mice that subsequently received 4 consecutive daily immunizations with HspE7 plus polyICLC started to regress these very large, established tumors, generally within one week of starting the 4 consecutive daily dose immunization regimen. Tumor volume was measured daily during the treatment period and then every 2 to 3 days thereafter. Tumors were measured using an electronic digital caliper (Fowler Sylvac Ultra-Cal Mark III) and were calculated by width²×length×0.5. Tumors continued to regress for 17 days following treatment in the majority (7 of 9) mice. In mice demonstrating re-emerging tumors, only escape variants were represented, no longer expressing the E7₄₉₋₅₇ epitope (data not shown). Mice that received 4 consecutive daily doses of buffer only, HspE7 protein only or polyICLC adjuvant only exhibited no regression of these large tumors.

### Example 12

### Boosting Effect of Repeat Immunizations During the Expansion Phase of a CD8 Response

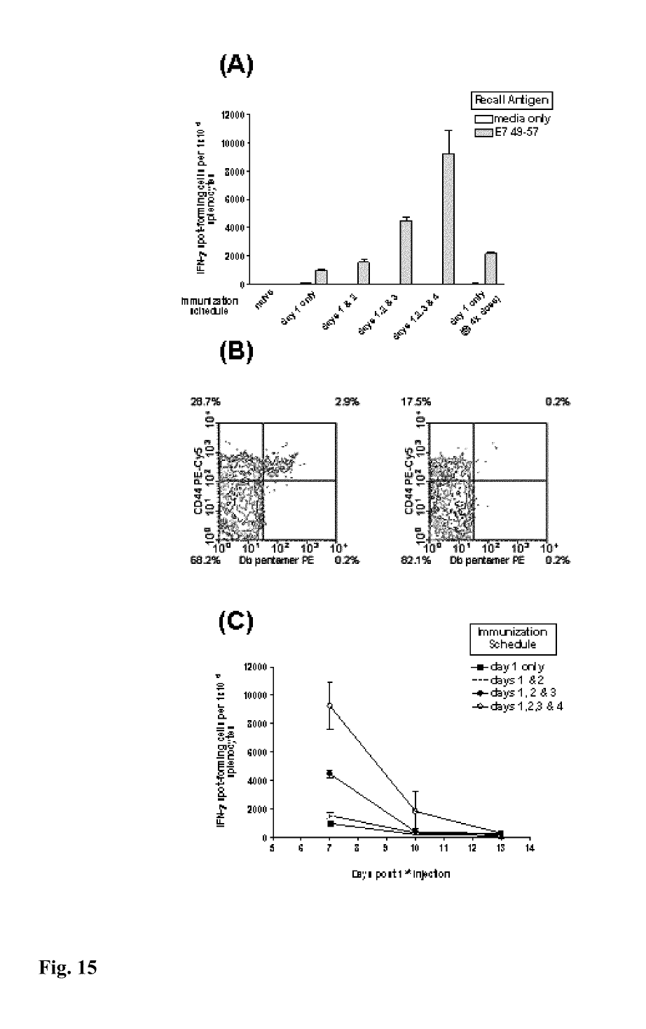

When mice were immunized with HspE7 plus polyICLC for one, two, three or four consecutive days, the levels of E7₄₉₋₅₇-specific T cells elicited underwent a dramatic increase after each subsequent daily immunization (FIG. 15A). After 4 successive daily immunizations with 100 ug HspE7 plus 10 ug polyICLC, the number of cells producing IFN-gamma directly ex vivo in response to stimulation with E7₄₉₋₅₇ approached 10,000 per 10⁶ splenocytes. Indeed for accurate IFN-gamma ELISPOT quantitation, splenocytes from immune animals had to be diluted 1:16 with splenocytes from naïve animals in order for the spots to be reduced to a number that was ‘countable’ by automated ELISPOT reader. This is approximately 10-fold the number of antigen-specific cells observed in animals receiving a single dose of HspE7 plus polyICLC. What was even more surprising was that these very high numbers of antigen-specific cells were reached within 3 days of the last immunization (all groups of mice were analyzed at 7 days after the first immunization). The four successive immunizations does not merely represent an additive increase in the amount of antigen mice were exposed to as mice given a single immunization containing 4 times the amount of antigen/adjuvant present in the single immunization had an increase in the number of E⁷₄₉₋₅₇-specific T cells but were still far below the numbers of E7₄₉₋₅₇-specific T cells observed in mice receiving 4 consecutive immunizations (FIG. 15A). E7₄₉₋₅₇-specific T cells were also readily detectable by flow cytometry using H-2Db/E7₄₉₋₅₇ pentamer reagents (FIG. 15B). After four consecutive daily immunizations with HspE7 plus polyICLC the number of E7₄₉₋₅₇-specific T cells in some animals reached as high as 2.9% of the total number of CD8⁺ splenocytes. Flow cytometric quantitation of E7-specific T cells with MHC class I pentamer reagents somewhat underestimated the number of antigen-specific cells as compared to ELISPOT, however, this is likely a reflection of down-regulation of surface TCR on antigen-specific T cells, particularly as the flow cytometric analysis was performed only three days after the last of four successive immunizations. Indeed, closer inspection of the flow cytometric data shown in FIG. 5B confirms that there are much higher numbers of CD8⁺ cells that are H-2Db/E7₄₉₋₅₇ pentamer-negative but which express the CD44 activation marker in mice receiving 4 successive daily immunizations compared to naïve mice. These CD8⁺ cells with an activated phenotype likely correspond to antigen-specific cells that have down-regulated their surface TCR as a result of their in vivo activation state. In addition, we also analyzed the contraction phase of the immune response to assess whether immunization on multiple consecutive days had a significant impact on the duration of the ensuing immune response. As shown in FIG. 15C, despite the large differences in the peak immune responses observed at day 7 post-immunization, E7-specific CD8⁺ cell numbers underwent significant contraction by day 13 post-immunization in all mice regardless of the number of consecutive immunizations on days 1 through 4. However, it should be noted that E7-specific CD8⁺ cells were still readily detectable by ELISPOT at day 13 post-immunization, and more importantly, that higher antigen-specific T cell numbers at the peak of the primary response correlated with the residual numbers of E7-specific CD8⁺ cells observed at day 13.

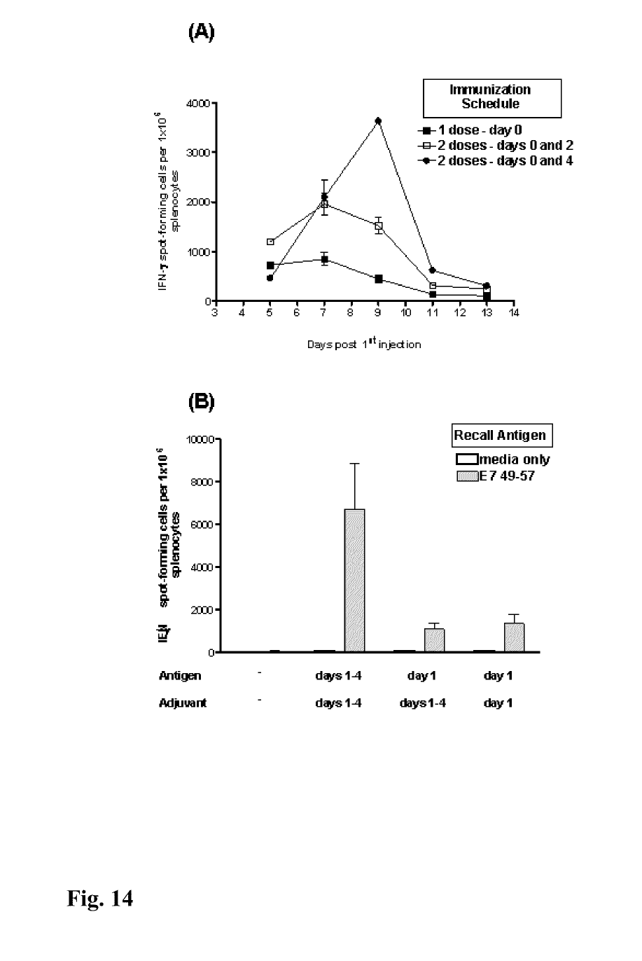

The effect of two injections given within the expansion phase of the response but with varying intervals between the first and second injection with respect to augmentation of the ensuing primary CD8 T cell response was investigated. Spleens were harvested at varying intervals during the study to investigate the kinetics of the ensuing response. Mice given a single injection of HspE7 plus polyICLC mounted a response that was readily detectable five days after immunization and which subsequently peaked at day 7 post-immunization (FIG. 14A). This response was in decline by day 9 and had essentially waned to a low (but stable) and readily detectable level by day 11. In contrast, mice given a primary immunization of HspE7 plus polyICLC on day 0 and then a second identical immunization on day 2 mounted a response that was much stronger than that elicited in mice receiving a single immunization. As was observed in mice receiving a single immunization, the CD8 T cell response was maximal on day 7 but reached a significantly higher overall number of antigen-specific cells. Furthermore, although the number of antigen-specific cells was in decline by day 9, the overall number of antigen-specific cells present at this time remained significantly higher than what was observed in mice receiving a single immunization. When the second immunization was delayed to day 4 after the primary immunization the effect was even more striking. In this case the number of antigen-specific T cells continued to rise through day 7 and did not reach a maximum until day 9, at which time the frequency of antigen-specific cells was approximately 4-fold the maximum number observed after a single immunization.

It was observed that a single dose of HspE7 plus polyICLC followed by three consecutive doses of polyICLC alone did not elicit a significant increase in the numbers of E7-specific CD8⁺ cells compared to mice receiving a single dose of HspE7 plus polyICLC (FIG. 14B). This result suggests that the dramatic expansion of E7-specific CD8⁺ cells elicited in mice receiving consecutive daily immunizations was riot simply an indirect consequence of the continuous presence of adjuvant, but was dependent upon the presence of specific antigen.

### Example 13

### Phase 1 Safety Study of HspE7 and Poly-ICLC Administered Concomitantly in Cervical Intraepithelial Neoplasia (CIN) Subjects

The primary objective of this study was to demonstrate safety and tolerability of concomitant administration of the heat shock protein fused HPV 16 E7 antigen (HspE7) and the TLR-3 agonist Poly ICLC in women with cervical intraepithelial neoplasia (CIN). Secondary objectives include the evaluation of immunologic parameters to characterize the immune response against HspE7 at various intervals throughout the study. A multicenter, nonrandomized, open label, Phase 1 study was designed to evaluate the safety and tolerability of escalating doses of Poly ICLC on a fixed dose of HspE7. Patients were immunized with 3 doses subcutaneously, once every 28 days in the upper thigh and then followed for 1 month after the last immunization. Adjuvant dose escalation was performed in the following cohorts:
Cohort 1: 500-μg HspE7+50-μg Poly-ICLC in 4 subjects
Cohort 2: 500-μg HspE7+500-μg Poly-ICLC in 4 subjects
Cohort 3: 500-μg HspE7+1000-μg Poly-ICLC in 4 subjects
Cohort 4: 500-μg HspE7+2000-μg Poly-ICLC in 4 subjects

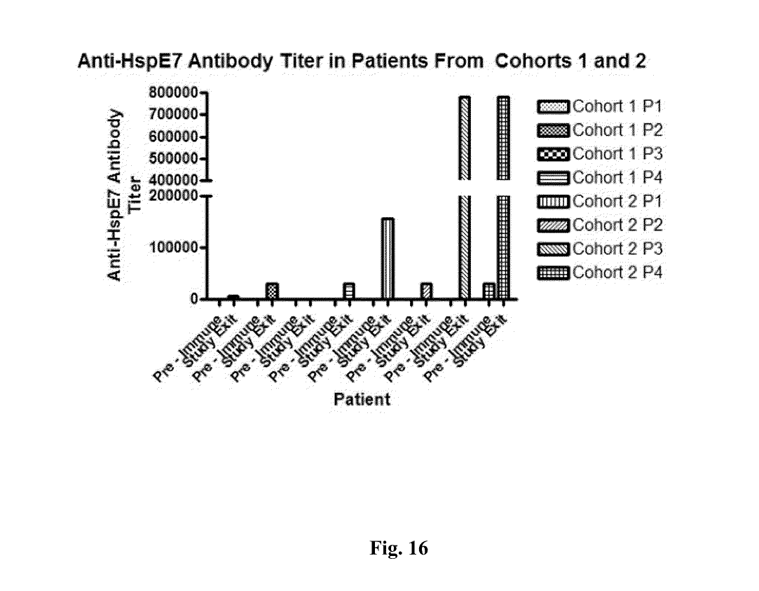

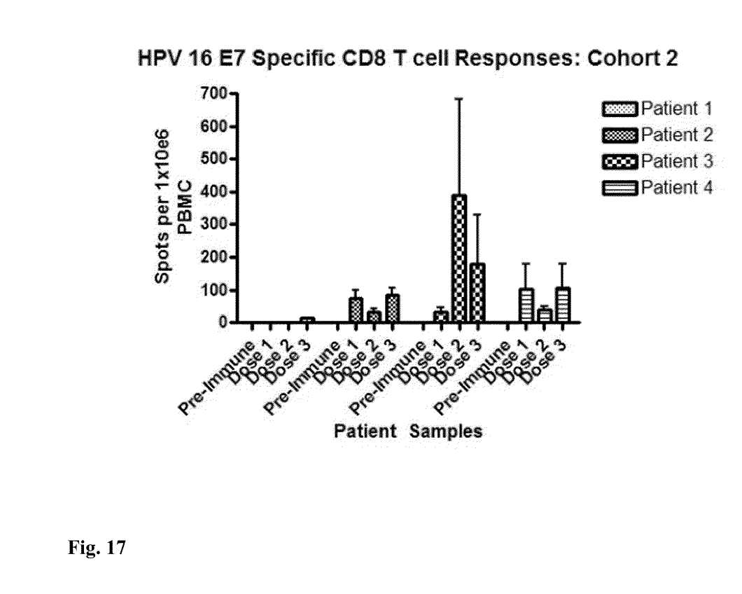

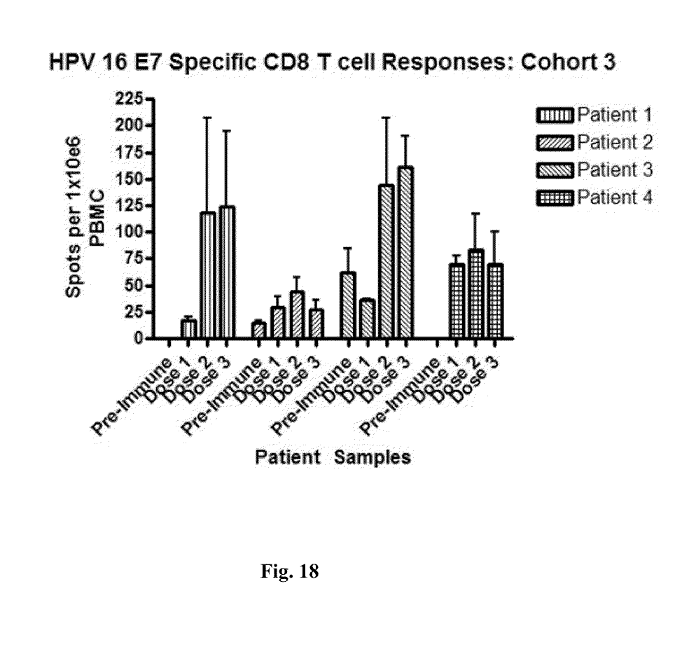

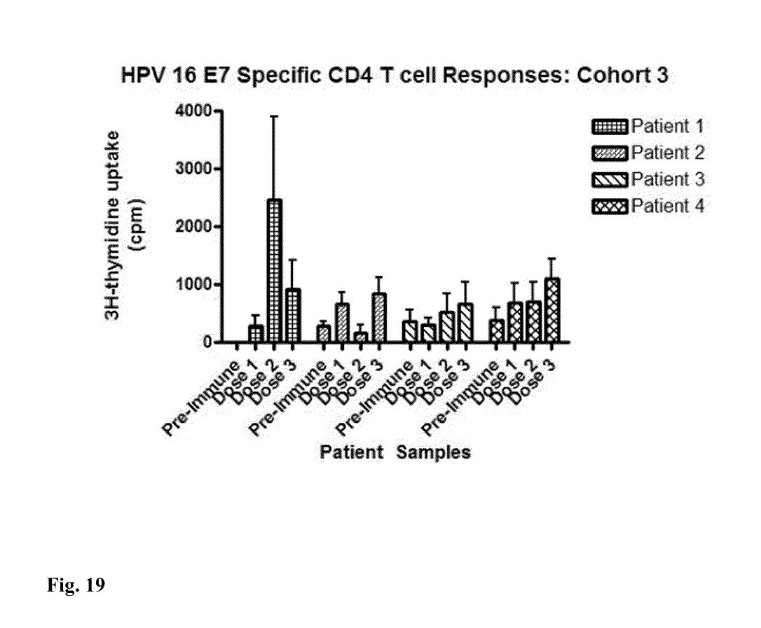

Immunological monitoring was performed on blood draws taken prior to the first immunization to establish a baseline for comparison and 7 days after each administration of HspE7 and Poly ICLC. These samples were analyzed for cellular immune responses and antibody responses. HspE7 and Poly ICLC were determined to be safe and well tolerated in all cohorts. Minor injection site reactions and flu like symptoms were seen in all cohorts with the number of patients per cohort experiencing these symptoms increasing with the escalating dose of Poly ICLC. Immunologically, patients demonstrated significant changes in antibody responses to HspE7 with all showing an increased antibody titer to the immunizing antigen (FIG. 16). T cell responses demonstrated a dose response with the Cohort 1 showing no responses and Cohorts 2 & 3 demonstrating clear CD4 and CD8 HPV 16 E7 antigen specific immune responses (FIG. 17-19).

In conclusion, the Phase 1 demonstrated that the combination of HspE7 and Poly ICLC is safe, tolerable and immunogenic in humans; showing antibody and T cell responses to the immunizing antigen.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A method of increasing the biological activity of purified HspE7 comprising, administering the HspE7 along with an immune stimulant selected from the group consisting of CpG containing oligonucleotides, a TLR3 agonist, mono-phosphoryl-lipid A (MPL), MPL-trehalose 6,6′-dimycolate (MPL-TDM), and anti-CD40.

2. The method of claim 1, wherein the immune stimulant is present at an amount from about 0.1 ug to about 20 mg/ml.

3. The method of claim 1, wherein the purified HspE7 is of a purity of about 95% to about 99.99% HspE7 as determined using 1% PAGE.

4. The method of claim 1 wherein the immune stimulant is a TLR3 agonist.

5. The method of claim 4, wherein the TLR3 agonist is polyICLC or polyI:C.

6. A composition comprising purified HspE7 and an immune stimulant selected from the group consisting of CpG-containing oligonucleotides, a TLR3 agonist, mono-phosphoryl-lipid A (MPL), MPL-trehalose 6,6′-dimycolate (MPL-TDM), and anti-CD40.

7. The composition of claim 6, wherein the immune stimulant is present at an amount from about 0.1 ug to about 20 mg/ml.

8. The composition of claim 6, wherein the purified HspE7 is of a purity of about 95% to about 99.99% HspE7 as determined using 1% PAGE.

9. The composition of claim 6 wherein the immune stimulant is a TLR3 agonist.

10. The composition of claim 9, wherein the TLR3 agonist is polyICLC or polyI:C.

11. A method of reducing tumor or virus development in a subject comprising administering the composition of claim 6 to the subject in need thereof.

12. A kit comprising purified HspE7 and an immune stimulant selected from the group consisting of CpG-containing oligonucleotides, a TLR3 agonist, mono-phosphoryl-lipid A (MPL), MPL-trehalose 6,6′-dimycolate (MPL-TDM), and anti-CD40, and instruction for use.

13. The kit of claim 11, wherein the immune stimulant is present at an amount from about 0.1 ug to about 20 mg/ml, and the purified HspE7 is of a purity of about 95% to about 99.99% HspE7 as determined using 1% PAGE.

14. The kit of claim 11 wherein the immune stimulant is a TLR3 agonist.

15. The kit of claim 11, wherein the TLR3 agonist is polyICLC or polyI:C.

16. Use of the composition of claim 6 for the prevention or treatment of cancer in a subject in need thereof.

17. Use of the composition of claim 6 for the reducing tumor or virus development in a subject in need thereof.

18. The method of claim 11, wherein said composition is administered according to a dosing schedule comprising at least two doses.

19. A method of preventing tumor or virus development in a subject, comprising administering the composition of claim 6 to the subject in need thereof.
